(19) United States
(12) Patent Application Publication
(10) Pub. No.: US 2012/0034616 A1
(43) Pub. Date: Feb. 9, 2012

(54) Method for Modulating Epithelial Stem Cell Lineage

(75) Inventors: Elaine Fuchs, New York, NY (US); Colin Jamora, San Diego, CA (US); Charles K. Kaufman, Brookline, MA (US); Krzysztof Kobielak, New York, NY (US)

(73) Assignee: The Rockefeller University, New York, NY (US)

(21) Appl. No.: 12/894,720

(22) Filed: Sep. 30, 2010

Related U.S. Application Data
(63) Continuation of application No. 10/544,999, filed on Dec. 30, 2005, now abandoned.
(60) Provisional application No. 60/450,923, filed on Feb. 27, 2003.
(60) Provisional application No. 60/494,479, filed on Aug. 12, 2003.
(60) Provisional application No. 60/516,918, filed on Nov. 3, 2003.

(51) Int. Cl. C12Q 1/68; G01N 33/566; C12N 5/071

(52) U.S. Cl. 435/6.13; 435/375; 435/7.1

(57) ABSTRACT

The present invention relates to methods of modulating epithelial stem cell lineage by regulating the expression of Lef1 or a BMP inhibitor and/or the stability of β-catenin or the expression of a Wnt; regulating the expression or activity of GATA-3; or regulating BMPR1A activity either at the level of receptor expression or at the level of pathway activation. Methods of regulating E-cadherin, GATA-3, BMPR1A and HK1-hair keratin and methods of identifying agents which modulate the epithelial stem cell lineage are further provided. Such agents are useful for inhibiting or stimulating inner root sheath development or hair follicle formation.

### INTRODUCTION

This invention was made with government support under Grant Numbers 5 T32 GM07281 and R01-AR31737 awarded by the National Institutes of Health. The government has certain rights in the invention.

### BACKGROUND OF THE INVENTION

During embryonic development, a layer of multipotent stem cells gives rise to epidermis and its appendages, including hair follicles. Hair follicle morphogenesis arises from a series of epithelial-mesenchymal cues, initiating an epithelial downgrowth which then proliferates and differentiates to form first the channel, or inner root sheath (IRS), and then the hair itself. Postnatally, the mature hair shaft is composed of a core, or medulla, cloaked by a concentric ring of cortical cells, which in turn are surrounded by a layer of hair shaft cuticle (the surface of the hair). Beneath the skin surface, the hair shaft is surrounded by the IRS, which is composed of a cuticle, Huxley's and Henle's layers. The IRS cuticle cells interlock with the hair cuticle cells, but near the skin surface, the IRS degenerates to release the shaft (Dry (1926) Genetics 16:287-340; Hardy (1992) Trends Genet. 8:55-61). Above the base (bulb) of the follicle, the Henle's layer is encased by the companion layer and outer root sheath (ORS), a structure contiguous with and biochemically similar to the epidermal basal layer.

Both hair shaft and IRS form concomitantly through upward terminal differentiation of rapidly dividing progenitor (matrix) cells, which maintain contact with the dermal papilla, the follicular mesenchyme located at the core of the hair bulb (Oliver and Jahoda (1988) Clin. Dermatol. 6:74-82; Cotsarelis et al. (1990) Cell 61:1329-1337). It is thought that a reservoir of stem cells, the bulge, resides at the base of the non-cycling segment of the hair follicle and replenishes the matrix cells as they differentiate (Cotsarelis et al. (1990) supra). Contact with dermal papilla cells appears to be essential, although not necessarily sufficient, to convert stem cells to transiently amplifying matrix cells (Taylor et al. (2000) Cell 102(4):451-61; Oshima et al. (2001) Cell 104:233-245).

The spatially defined differentiation programs of the hair follicle provides a system for studying the molecular mechanisms that underlie epithelial-mesenchymal cross-talk during stem cell lineage determination. A myriad of signal transduction pathways, including TGF-βs, Bone morphogenetic proteins (Burps), Sonic hedgehog (Shh), and Wnts, provide external cues that orchestrate cell fate decisions during hair follicle morphogenesis (Fuchs et al. (2001) Dev. Cell 1:13-25; Panteleyev et al. (2001) Cell Sci. 114:3419-3431; Millar (2002) J. Invest. Dermatol. 118:216-225; Niemann and Watt (2002) Trends Cell Biol. 12:185-192). Sonic hedgehog (Shh) is expressed in a subset of matrix cells and is thought to play a role in follicle proliferation (Oro and Higgins (2003) Dev. Biol. 255:238-248). Hair shaft differentiation is dependent upon Wnt signaling, and alterations in this pathway generate a number of follicle abnormalities (Gat et al. (1998) Cell 95:605-614; Chan et al. (1999) Nat. Genet. 21:410-413; Millar et al. (1999) Dev. Biol. 207133-149; Huelsken et al. (2001) Cell 105:533-545; Andl et al. (2002) Dev. Cell 2:643-653; Millar (2002) supra; Alonso and Fuchs (2003) Genes Dev. 17:1189-1200). In mice, the Wnt reporter gene TOPGAL is most highly active in the differentiating cortical cells that have withdrawn from the cell cycle (DasGupta and Fuchs (1999) Development 126:4557-4568).

Canonical Wnt signaling results in the stabilization of β-catenin, which activates members of the Lef1/Tcf family of DNA binding proteins (Moon et al. (2002) Science 296:1644-1646). In order for matrix cell progeny to respond to Wnt signaling (Reddy et al. (2001) Mech. Dev. 107:69-82), they must express Lef1. It has been shown that Lef1 expression in keratinocytes is dependent upon Noggin, a dermal papilla-secreted inhibitor of BMP signaling (Botchkarev et al. (1999) Nat. Cell Biol. 1:158-64). When Lef1-positive, hair progenitor cells respond to Wnts, they activate the hair-specific keratin genes, which possess Lef1 DNA binding sites in their 5′ regulatory regions (Zhou et al. (1995) Genes Dev. 9:700-713; Merrill et al. (2001) Genes Dev. 15:1688-1705). Although Lef1/β-catenin complexes appear to be required for hair keratin gene expression, other transcription factors, including FoxN1 and Hoxc13, may also be involved (Prowse et al. (1999) Dev. Biol. 212:54-67).

The role of BMP inhibition in the hair follicle is unclear. Lef1 mRNA expression is highest in proliferating matrix cells, and there is an unexplained lag before signs of Wnt responsiveness occur, followed by concentration of Lef1 and β-catenin in the nucleus and progression toward hair differentiation (Zhou et al. (1995) supra; DasGupta and Fuchs (1999) supra). Moreover, a positive role for BMPs has also been postulated in the follicle, where both proliferation and differentiation seem to be affected by BMP activation (Blessing et al. (1993) Genes Dev. 7:204-215; Blessing et al. (1994) J. Cell Biol. 135:227-239). Additionally, while Noggin has a positive effect on Lef1 expression, it has a negative effect in cortex, where ectopic expression of Noggin diminishes FoxN1 and Hoxc13 expression and blocks hair differentiation (Kulessa et al. (2000) EMBO J. 19:6664-6674).

The difficulties in understanding BMP-mediated regulation in the follicle are compounded by the complex expression patterns involved. BMP2 and BMP4 are in epithelial hair progenitor cells, BMP4 and BMP7 are in the dermal papilla, BMP7 is in the ORS, and BMP7, BMP8a and BMP8b are in the IRS (Zhao and Hogan (1996) Mech Dev. 57:159-168; Takahashi and Ikeda (1996) Dev. Dyn. 207:439-449; Kratochwil et al. (1996) Genes Dev. 10:1382-1394; Wilson et al. (1999) Exp. Dermatol. 8:367-368).

Thus, understanding the signal transduction pathways involved in follicle formation provides targets for stimulating or inhibiting hair growth.

### SUMMARY OF THE INVENTION

The present invention relates to methods for modulating epithelial stem cell lineage by regulating the expression or activity of polypeptides involved in this process. The methods of the invention involve regulating the expression of Lef1 or a BMP inhibitor in combination with regulating the stability of β-catenin or the expression of a Wnt; regulating the expression or activity of GATA-3; or regulating the activity of bone morphogenetic protein receptor IA. Increasing the expression of Lef1 or a BMP inhibitor and the stability of β-catenin or the expression of a Wnt decreases E-cadherin expression; increases the expression of HK1-hair keratin; and stimulates epithelial bud formation. Stimulating epithelial bud formation is useful, for example, in promoting hair growth. Decreasing the expression of Lef1 or a BMP inhibitor and the stability of β-catenin or the expression of a Wnt reduces epithelial bud formation and is useful, for example, in inhibiting hair growth and development of cancer. In particular embodiments, the Wnt is Wnt1, Wnt2, Wnt 3a, Wnt8a, Wnt 8b, or Wnt10 and the BMP inhibitor is noggin, gremlin or chordin.

The present invention further relates to methods for identifying an agent which modulates epithelial stem cell lineage by utilizing the polypeptides involved in this process. Screening methods of the invention involve contacting a test cell, which contains a reporter operably linked to an E-cadherin promoter sequence; GATA-3 promoter sequence or a promoter sequence containing a GATA-3 binding site; or BMPR1A promoter sequence, with at least one agent and detecting expression of a product of the nucleic acid sequence encoding the reporter in the test cell. In particular embodiments, cells containing a reporter operably linked to an E-cadherin promoter sequence further contain nucleic acid sequences encoding a Wnt, a BMP inhibitor, Lef1, β-catenin. In one embodiment, a decrease in the expression of a product of the nucleic acid sequence encoding the reporter in the test cell contacted with the agent relative to the expression of the product of the nucleic acid sequence encoding the reporter in a test cell not contacted with the agent, indicates that the agent causes a decrease in expression of a product of the nucleic acid sequence encoding E-cadherin, GATA-3 or BMPR1A in the test cell. In another embodiment, an increase in the expression of a product of the nucleic acid sequence encoding the reporter in the test cell contacted with the agent relative to the express'ion of the product of the nucleic acid sequence encoding the reporter in a test cell not contacted with the agent, indicates that the agent causes an increase in expression of a product of the nucleic acid sequence encoding E-cadherin, GATA-3 or BMPR1A in the test cell. Agents identified in these screening methods of the invention are useful in for stimulating inner root sheath or hair shaft development or stimulating or inhibiting epithelial bud formation to modulate the development of hair follicles, teeth, lungs and the like.

In an alternative embodiment of the screening methods of the invention, an agent which modulates inner root sheath or hair shaft formation is identified by contacting a test cell, which contains or lacks a functional morphogenetic protein receptor IA, with an agent and detecting the phosphorylation state of Smad-1 in the test cell.

In a further embodiment of the screening methods of the invention, an agent which modulates inner root sheath or hair shaft formation is identified by contacting a test cell lacking functional BMPR1A and containing a nucleic acid sequence encoding a reporter operably linked to a Wnt-responsive promoter with a test agent and detecting expression of the reporter in the test cell. An increase in the expression of the reporter in the test cell contacted with the agent relative to the expression of the reporter in a test cell not contacted with the agent indicates that the agent increases expression of a Wnt-responsive gene to stimulate hair shaft development.

### DETAILED DESCRIPTION OF THE INVENTION

During follicular morphogenesis, stem cells form a bud structure by changing their polarity and cell-cell contacts. It has now been found that this process is achieved through simultaneous receipt of at least two external signals: a Wnt to stabilize β-catenin, and a BMP inhibitor to produce Lef1. β-catenin-activated Lef1 transcription complexes then appear to act uncharacteristically by downregulating the gene encoding E-cadherin, a key component of polarity and intercellular adhesion. When either signal is missing, functional Lef1 complexes are not made, and E-cadherin downregulation and follicle formation is impaired. Noggin and Wnt are an additional level of regulation in the process. Further, BMP receptor 1A (BMPR1a) is essential for the morphological and biochemical differentiation of transiently dividing progenitor cells of the inner root sheath and hair shaft. Moreover, it has now been found that GATA-3 is essential for stem cell lineage determination in skin, where it is expressed at the onset of epidermal stratification and IRS specification in follicles. GATA-3 null/lacZ knockin embryos survived up to E18.5 where they failed to form the IRS. Skin grafting studies demonstrated additional defects in GATA-3 null hairs and follicles. IRS progenitors failed to differentiate, while cortical progenitors differentiated, but produced an aberrant hair structure. Some GATA-3 null progenitor cells expressed mixed IRS and hair shaft markers. Taken together, these findings indicate that GATA-3 with Lef-1/Wnts are at the crossroads of the IRS versus hair shaft cell fate decision in hair follicle morphogenesis and BMPR1a is essential to this process.

The results provided herein indicate that embryonic skin epithelial stem cells require simultaneous inputs of stimulatory and inhibitory signals from multiple neighboring cell types for the purpose of producing an activated transcription factor able to remodel adherens junction (AJ) gene expression and form a follicle bud. Such a system furnishes a level of governance by converging signals to specify stem cell lineage.

In one embodiment of the present invention, two external signals: a Wnt, to stabilize β-catenin, and a BMP inhibitor, to produce Lef1, are necessary to regulate the expression of E-cadherin, a key protein in epithelial stem cell lineage. Accordingly, the present invention provides a method of modulating epithelial stem cell lineage by regulating the expression of Lef1 or a BMP inhibitor in combination with regulating the stability of β-catenin or the expression of a Wnt. As used herein, Lef1 is meant to include homologs of Lef1 which bind stabilized β-catenin to form transcription complexes capable of regulating the expression of E-cadherin. Decreasing Lef1 or BMP inhibitor expression and the expression of a Wnt or stability of β-catenin is useful in reducing or inhibiting epithelial bud formation to decrease, for example, unwanted hair growth or cancer development. Increasing Lef1 or BMP inhibitor expression and expression of a Wnt or β-catenin stability is useful in promoting or stimulating epithelial bud formation to produce, for example, hair follicles. Means of regulating the expression of Lef1, a Wnt, a BMP inhibitor and the stability of β-catenin are provided herein.

In another embodiment of the present invention, E-cadherin expression is decreased by administering an effective amount of a BMP inhibitor or Lef1 in combination with a Wnt or stabilized β-catenin so that E-cadherin expression is decreased. It is contemplated that any combination (i.e., a Wnt and a BMP inhibitor; a Wnt and Lef1; stabilized β-catenin and a BMP inhibitor; or stabilized β-catenin and Lef1) may be administered to decrease the expression of E-cadherin. E-cadherin expression may be determined as exemplified herein or using any other means of detecting an RNA transcript (e.g., reverse transcriptase PCR, real-time RCR, in situ hybridization, northern blot analysis, microarray) or protein (e.g., immunoassays or proteomic approaches).

Results provided herein further demonstrate that HK1-hair keratin expression is also regulated by Wnt and BMP inhibitor levels. Thus, in a further embodiment of the present invention, HK1-hair keratin expression is increased by administering an effective amount of a Wnt and BMP inhibitor. As HK1-hair keratin is the major structural protein in the hair shaft this embodiment is useful in increasing the strength of hair.

Wnts useful in carrying out the aforementioned methods of the invention include, but are not limited to, Wnt1, Wnt2, Wnt3a, Wnt8a, Wnt 8b, or Wnt10 and the like. In one embodiment of the present invention, a Wnt comprises Wnt1 or Wnt3a. Exemplary BMP inhibitors include, but are not limited to, noggin, gremlin or chordin. In another embodiment of the present invention, a BMP inhibitor is noggin. Further, β-catenin can be stabilized using a Wnt or via recombinant engineering to produce a constitutively stable β-catenin (e.g., K14ΔNβ-catenin). Nucleic acid and proteins sequences of Wnts, BMP inhibitors, and β-catenins are well-established in the art and are readily available in public databases such as EMBL and GENEBANK.

GATA-3 has now been shown to regulate epithelial stem cell lineage, in particular IRS differentiation. Accordingly, a further embodiment of the present invention provides a method for modulating epithelial stem cell lineages by regulating the expression or activity of GATA-3. Decreasing GATA-3 expression or activity is useful in reducing or inhibiting inner root sheath development to decrease or inhibit, for example, unwanted hair growth or alter the structure of hair (e.g., awl, zig-zag, guard, and the like). Increasing GATA-3 expression or activity is useful in promoting or stimulating inner root sheath development to promote or stimulate, for example, hair follicle formation or to alter the structure of hair. It is contemplated that altering the structure of hair will be useful in wool production or for cosmetic concerns such as an individuals desire to have straighter, curlier or thicker hair. Means of modulating the expression or activity of GATA-3 are provided herein.

The results provided herein show that BMPR1A receptor is essential for stem cell lineage determination. Accordingly, the present invention provides a method for modulating inner root sheath and/or hair shaft development via increasing or decreasing the level of expression or activity of a BMPR1A receptor. The level of activity of a BMPR1A receptor can be altered by changing the expression of the BMPR1A receptor (i.e., increasing the amount of BMPR1A receptor present in a cell) or changing the activity (i.e., the ability of the receptor either to bind ligand or transmit a signal) of the BMPR1A receptor. Decreasing BMPR1A receptor expression or activity is useful in reducing or inhibiting inner root sheath and/or hair shaft formation to decrease or inhibit, for example, unwanted hair growth. Increasing BMPR1A expression or activity could be useful in promoting or stimulating inner root sheath and/or hair shaft formation to promote or stimulate hair growth (e.g., in thinning or balding individuals). In general, BMPR1A receptor activity can be modulated by exogenously supplying an agent which increases or decreases the expression or activity of a BMPR1A receptor or modulates a BMPR1A receptor signaling pathway.

Methods of modulating epithelial stem cell lineage or inner root sheath and/or hair shaft development provided heretofore may be carried out via genetic engineering (gene therapy), by administering agents which modulate the expression or activity of the polypeptides which regulate said processes (i.e., a BMP inhibitor, Lef1, a Wnt, stabilized β-catenin, GATA-3, or BMPR1A receptor, referred to hereafter as polypeptides of the invention) or by administering a purified recombinant polypeptide of the invention (e.g., encapsulated in liposome formulations) to effectively increase the activity of a polypeptide of the invention. Thus, the present invention provides methods for genetically engineering a cell to express a polypeptide of the invention, methods for preparing and administering a polypeptide of the present invention, and methods for identifying or screening for agents with modulate the expression or activity of a polypeptide of the invention.

In one embodiment, genetic engineering or gene therapy approaches to modulating the expression of a polypeptide of the invention may include either increasing or decreasing the expression of said polypeptide in a cell to promote or reduce hair follicle formation. Any genetic engineering method known in the art can be used to decrease or increase expression of a polypeptide of the invention. Methods of decreasing expression of a polypeptide of the invention in a cell via genetic engineering include, but are not limited to, the use of inhibitory RNA molecules (e.g., ribozymes, RNAi, siRNA or antisense, collectively referred to herein as RNA molecules) or targeted gene disruption. A method for increasing expression of a polypeptide of the invention via genetic engineering includes, but is not limited to, providing nucleic acid sequences encoding a polypeptide of the invention to a cell in need of increased levels of a polypeptide of the invention.

To generate a targeted gene disruption or knock-out at the locus encoding a polypeptide of the invention, a transgene is typically inserted within or adjacent to the coding region for the polypeptide. A transgene is meant to refer to heterologous nucleic acid that, upon insertion in or near the locus encoding a polypeptide of the invention, results in a decrease in gene expression or inactivation of said locus.

A knock-out of a locus encoding a polypeptide of the invention means an alteration in the nucleotide sequence of said locus that results in a decrease, reduction, or elimination of messenger RNA encoding the polypeptide or decrease or a decrease, reduction, or elimination of the amount or activity of the polypeptide. Knock-outs as used herein also include conditional knock-outs, where alteration of the locus encoding the polypeptide of the invention may occur upon, for example, exposure of a cell or animal containing said locus to a substance that promotes gene alteration or introduction of an enzyme that promotes recombination at the locus encoding the polypeptide of the invention (e.g., Cre in the Cre-lox system).

In general, a knock-out construct is a nucleic acid sequence, such as a DNA construct, which, when introduced into a cell, results in suppression (partial or complete) of expression of a polypeptide or protein encoded by endogenous DNA in the cell. A knock-out construct as used herein can include a construct containing a first fragment from the 5′ end of the locus encoding a polypeptide of the invention, a second fragment from the 3′ end of the locus encoding a polypeptide of the invention and a DNA fragment encoding a selectable marker positioned between the first and second fragments. It should be understood by the skilled artisan that any suitable 5′ and 3′ fragments of the locus encoding a polypeptide of the invention can be used so long as the expression of the corresponding polypeptide of the invention is partially or completely suppressed by insertion of the transgene. Suitable selectable markers include, but are not limited to, the well-established proteins which confer resistance to neomycin, puromycin and hygromycin.

Expression levels of a polypeptide of the invention can also be altered using an antisense oligonucleotide sequence. The antisense sequence is complementary to at least a portion of the coding sequence of polypeptide of the invention or mRNA sequence of polypeptide of the invention (e.g., hybridizing to the coding sequence or 5′- or 3′-untranslated regions). The coding sequence and untranslated regions of a polypeptide of the invention are readily available from databases such as Genbank (e.g., GATA-3 is found at accession numbers X55122, X55037, AJ131811 and SEQ ID NO:1; BMPR1A is found at accession numbers NM_{—}004329 and 223154 for H. sapiens and M. musculus, respectively and SEQ ID NO:2). Antisense oligonucleotide sequences are at least 11 nucleotides in length, but can be at least 12, 15, 20, 25, 30, 35, 40, 45, or 50 or more nucleotides long and can be determined in accordance with routine procedures. Longer sequences can also be used. Antisense oligonucleotide molecules can be provided in a construct and introduced into cells as naked nucleic acids using standard methodologies to decrease expression of a polypeptide of the invention.

In another embodiment, RNA interference (RNAi) is used to modulate the expression and, hence, activity of a polypeptide of the invention. RNAi is a mechanism of post-transcriptional gene silencing in which double-stranded RNA (dsRNA) corresponding to a coding sequence of interest is introduced into a cell or an organism, resulting in degradation of the corresponding mRNA. The RNAi effect persists for multiple cell divisions before gene expression is regained. RNAi is therefore a powerful method for making targeted knockouts or knockdowns at the RNA level. RNAi has proven successful in human cells, including human embryonic kidney and HeLa cells (see, e.g., Elbashir, et al. (2001) Nature 411:494-8). In one embodiment, silencing can be induced in mammalian cells by enforcing endogenous expression of RNA hairpins (see, Paddison, et al. (2002) PNAS USA 99:1443-1448). In another embodiment, transfection of small (e.g., 21-23 nucleotide) dsRNA specifically inhibits nucleic acid expression (reviewed in Caplen (2002) Trends Biotech. 20:49-51).

The mechanism by which RNAi achieves gene silencing has been reviewed in Sharp, et al. (2001) Genes Dev 15:485-490; and Hammond, et al. (2001) Nature Rev. Gen. 2:110-119).

As with other embodiments of the invention, RNAi may be introduced via a gene therapy approach or alternatively utilizing standard molecular biology methods. For example, RNAi can be effected by the introduction of suitable in vitro synthesized siRNA or siRNA-like molecules into cells. RNAi can, for example, be performed using chemically-synthesized RNA. Alternatively, suitable expression vectors can be used to transcribe such RNA either in vitro or in vivo. In vitro transcription of sense and antisense strands (encoded by sequences present on the same vector or on separate vectors) can be effected using for example T7 RNA polymerase, in which case the vector can contain a suitable coding sequence operably-linked to a T7 promoter. The in vitro-transcribed RNA can in embodiments be processed (e.g., using E. coli RNase III) in vitro to a size conducive to RNAi. The sense and antisense transcripts are combined to form an RNA duplex which is introduced into a target cell of interest. Other vectors can be used, which express small hairpin RNAs (shRNAs) which can be processed into siRNA-like molecules. Various vector-based methods are described in for example Brummelkamp, et al. (2002) Science 296(5567):550-3; Lee, et al. (2002) Nat. Biotechnol. 20(5):500-5; Miyagashi and Taira (2002) Nat. Biotechnol. 20(5):497-500; Paddison, et al. (2002) Proc. Natl. Acad. Sci. USA 99(3):1443-8; Paul, et al. (2002); and Sui, et al. (2002) Proc. Natl. Acad. Sci. USA 99(8):5515-20. Various methods for introducing such vectors into cells, either in vitro or in vivo (e.g., gene therapy) are disclosed herein and well-known in the art.

Kits for production of dsRNA for use in RNAi are available commercially, e.g., from New England Biolabs, Inc. and Ambion Inc. (Austin, Tex., USA). Methods of transfection of dsRNA or plasmids engineered to make dsRNA are routine in the art.

An siRNA molecule can be specific for sequences in the 5′, 3′ or middle of the mRNA encoding a polypeptide of the invention. The target region can be selected experimentally or empirically. For example, siRNA target sites in a gene of interest are selected by identifying an AA dinucleotide sequence preferably in the coding region and not near the start codon (within 75 bases) as these may be richer in regulatory protein binding sites which can interfere with binding of the siRNA. (see, e.g., Elbashir, et al. (2001) Nature 411: 494-498). The subsequent 19-27 nucleotides 3′ of the AA dinucleotide can be included in the target site and in general have a G/C content of 30-50%.

Silencing effects similar to those produced by RNAi have been reported in mammalian cells with transfection of a mRNA-cDNA hybrid construct (Lin, et al. (2001) Biochem. Biophys. Res. Commun. 281:639-44), providing yet another strategy for silencing a coding sequence of interest.

In a further embodiment, a ribozyme is used to modulate the expression of a polypeptide of the invention. Ribozymes are RNA-protein complexes that cleave nucleic acids in a site-specific fashion, as is known in the art (e.g., U.S. Pat. No. 5,641,673). Ribozymes have specific catalytic domains that possess endonuclease activity (Kim, et al. (1987) Proc. Natl. Acad. Sci. USA 84:8788; Gerlach, et al. (1987) Nature 328:802; Forster and Symons (1987) Cell 49:211). For example, a large number of ribozymes accelerate phosphoester transfer reactions with a high degree of specificity, often cleaving only one of several phosphoesters in an oligonucleotide substrate (Michel and Westhof (1990) J. Mol. Biol. 216:585; Reinhold-Hurek and Shub (1992) Nature 357:173). This specificity has been attributed to the requirement that the substrate binds via specific base-pairing interactions to the internal guide sequence (IGS) of the ribozyme prior to chemical reaction. The nucleotide sequences of a polypeptide of the invention, as described herein, are sources of suitable hybridization region sequences.

Ribozyme catalysis has primarily been observed as part of sequence-specific cleavage/ligation reactions involving nucleic acids (Joyce (1989) Nature 338:217). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon, et al. (1991) Proc. Natl. Acad. Sci. USA 88:10591; Sarver, et al. (1990) Science 247:1222; Sioud, et al. (1992) J. Mol. Biol. 223:831).

RNA molecules of the present invention have a sufficient degree of complementarity to the mRNA encoding a polypeptide of the invention to avoid non-specific binding of the RNA molecule to non-target sequences under conditions in which specific binding is desired, such as under physiological conditions in the case of in vivo assays or therapeutic treatment or, in the case of in vitro assays, under conditions in which the assays are conducted. The target mRNA for RNA molecule binding can include not only the information to encode a protein, but also associated ribonucleotides, which for example form the 5′-untranslated region, the 3′-untranslated region, the 5′ cap region and intron/exon junction ribonucleotides. A method of screening for antisense, siRNA and ribozyme nucleic acids that can be used to provide such RNA molecules of the invention is disclosed in U.S. Pat. No. 5,932,435 (which is incorporated herein by reference).

Hybridization of RNA molecules provided herein to their cognate nucleic acid sequences encoding a polypeptide of the invention can be carried out under conditions of reduced stringency, medium stringency or even stringent conditions (e.g., conditions represented by a wash stringency of 35-40% Formamide with 5×Denhardt's solution, 0.5% SDS and 1×SSPE at 37° C.; conditions represented by a wash stringency of 40-45% Formamide with 5×Denhardt's solution, 0.5% SDS, and 1×SSPE at 42° C.; and/or conditions represented by a wash stringency of 50% Formamide with 5×Denhardt's solution, 0.5% SDS and 1×SSPE at 42° C., respectively) to the nucleotide sequences specifically disclosed herein. See, e.g., Sambrook et al., Molecular Cloning, A Laboratory Manual (2d Ed. 1989) (Cold Spring Harbor Laboratory).

Alternatively stated, an RNA molecule of the invention has at least about 60%, 70%, 80%, 90%, 95%, 97%, 98% or higher sequence similarity with the complement of the coding sequence of a polypeptide of the invention and will reduce the level of said polypeptide production.

In gene therapy approaches, knock-out constructs are used to express RNA molecules to decrease the expression of a polypeptide of the invention. Typically, for stable expression, the RNA molecule is placed under the control of a promoter. The promoter can be regulated, if deficiencies in the protein of interest may lead to a lethal phenotype, or the promoter can drive constitutive expression of the RNA molecule such that the gene of interest is silenced under all conditions of growth. While homologous recombination between the knock-out construct and sequences encoding the polypeptide of the invention may not be necessary when using an RNA molecule to decrease gene expression, it can be advantageous to target the knock-out construct to a particular location in the genome of the host organism so that unintended phenotypes are not generated by random insertion of the knock-out construct.

While the siRNA, ribozymes, or antisense RNA molecules can be expressed from knock-out constructs, isolated RNA molecules (oligonucleotides) of the invention can be introduced into a cell. To protect the RNA molecules from degradation, the RNA molecules can contain intersugar backbone linkages such as phosphotriesters, methyl phosphonates, short chain alkyl or cycloalkyl intersugar linkages or short chain heteroatomic or heterocyclic intersugar linkages, phosphorothioates and those with CH₂—NH—O—CH₂, CH₂—N(CH₃)—O—CH₂ (known as methylene(methylimino) or MMI backbone), CH₂—O—N(CH₃)—CH₂, CH₂—N(CH₃)—N(CH₃)—CH₂ and O—N(CH₃)—CH₂—CH₂ backbones (where phosphodiester is O—P—O—CH₂). Oligonucleotides having morpholino backbone structures cam also be used (U.S. Pat. No. 5,034,506). In alternative embodiments, oligonucleotides can have a peptide nucleic acid (PNA, sometimes referred to as protein nucleic acid) backbone, in which the phosphodiester backbone of the oligonucleotide is replaced with a polyamide backbone wherein nucleosidic bases are bound directly or indirectly to aza nitrogen atoms or methylene groups in the polyamide backbone (Nielsen, et al. (1991) Science 254:1497 and U.S. Pat. No. 5,539,082). The phosphodiester bonds can be substituted with structures which are chiral and enantiomerically specific. Persons of ordinary skill in the art will be able to select other linkages for use in practice of the invention.

Oligonucleotides can also include species which include at least one modified nucleotide base. Thus, purines and pyrimidines other than those normally found in nature can be used. Similarly, modifications on the pentofuranosyl portion of the nucleotide subunits can also be effected. Examples of such modifications are 2′-O-alkyl- and 2′-halogen-substituted nucleotides. Some specific examples of modifications at the 2′ position of sugar moieties which are useful in the present invention are OH, SH, SCH₃, F, OCN, O(CH₂)ₙNH₂ or O(CH₂)ₙCH₃ where n is from 1 to about 10; C₁ to C₁₀ lower alkyl, substituted lower alkyl, alkaryl or aralkyl; Cl; Br; CN; CF₃; OCF₃; O—, S—, or N-alkyl; O—, S—, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. One or more pentofuranosyl groups can be replaced by another sugar, by a sugar mimic such as cyclobutyl or by another moiety which takes the place of the sugar.

In particular embodiments, decreasing the expression of a polypeptide of the invention is intended to include decreasing mRNA or polypeptide amounts by 50%, 60%, 70%, or 80%. In other embodiments, expression of an mRNA or polypeptide of the invention is decreased by 90%, 95%, 99%, or 100%. Expression of an mRNA encoding a polypeptide of the invention can be assessed using methods well-known in the art, such as hybridization of nucleotide probes to mRNA, quantitative RT-PCR, or detection of the polypeptide of the invention using an antibody which specifically binds said polypeptide.

Any genetic engineering method known in the art can be used for enhancing or increasing expression of a polypeptide of the invention. For example, the coding sequence of a polypeptide of the invention can be delivered to cells in the vicinity of an area in which hair growth is desirable. Mechanical methods, such as microinjection, liposome-mediated transfection, electroporation, or calcium phosphate precipitation, can be used. Alternatively, if it is desired that the cells stably retain the construct, it can be supplied on a plasmid and maintained as a separate element or integrated into the genome of the cells, as is known in the art. The construct can include transcriptional regulatory elements, such as a promoter element, an enhancer or UAS element, and a transcriptional terminator signal, for controlling transcription of the coding sequence in the cells.

A construct can be directly introduced into a cell using well-known methods or incorporated into a viral vector for delivery to the host animal. Vectors, such as replication-defective retroviruses, adenoviruses and adeno-associated viruses can be used. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well-known to those skilled in the art. Examples of suitable packaging virus lines include ψCrip, ψCre, ψ2 and ψAm. The genome of adenovirus can be manipulated such that it encodes and expresses a polypeptide of the invention but is inactivated in terms of its ability to replicate in a normal lytic viral life cycle (Berkner, et al. (1988) BioTechniques 6:616; Rosenfeld, et al. (1991) Science 252:431-434; Rosenfeld, et al. (1992) Cell 68:143-155). Suitable adenoviral vectors derived from the adenovirus strain Ad type 5 dl324 or other strains of adenovirus (e.g., Ad2, Ad3, Ad7 etc.) are well-known to those skilled in the art. In vivo use of adenoviral vectors is described in Flotte, et al. ((1993) Proc. Natl. Acad. Sci. 90:10613-10617) and Kaplitt, et al. ((1994) Nature Genet. 8:148-153). Other viral vectors, such as those based on togaviruses or alpha viruses, can also be used. Alternatively, an adeno-associated virus vector such as that disclosed by Tratschin, et al. ((1985) Mol. Cell. Biol. 5:3251-3260) may be used to express a polypeptide of the invention.

A naked DNA construct encoding a polypeptide of the invention or an RNA molecule for decreasing the expression of a polypeptide of the invention can also be combined with a condensing agent, such as polylysine, polyarginine, polyornithine, protamine, spermine, spermidine, or putrescine, to form a gene delivery vehicle. Many suitable methods for making such linkages are known in the art. Alternatively, a construct encoding a polypeptide of the invention or an RNA molecule for decreasing the expression of a polypeptide of the invention can be associated with a liposome for delivery to a desired cell. Other suitable methods of providing such constructs or RNA molecules include DNA-ligand combinations, such as those disclosed in Wu, et al. ((1989) J. Biol. Chem. 264:16985-16987). Microbubble ultrasound transduction is another method for delivery of naked DNA (Lu, et al. (2003) Gene Ther. 10(5):396-405). Constructs or RNA molecules can also be delivered to the site of an internal cancer, for example, using receptor-mediated targeted delivery. Receptor-mediated DNA delivery techniques are well-known in the art (Findeis, et al. (1993) Trends Biotech. 11:202-05; Chiou, et al. (1994) Gene Therapeutics: Methods and Applications of Direct Gene Transfer (J. A. Wolff, ed.); Wu, et al. (1994) J. Biol. Chem. 269:542-46). Expression of a polypeptide of the invention can be monitored by detecting production of mRNA which hybridizes to a delivered coding sequence or by detecting the protein product of the gene using, for example, immunological techniques.

Expression of an endogenous polypeptide of the invention in a cell can also be altered by introducing in-frame with the endogenous gene a DNA construct comprising a targeting sequence, a regulatory sequence, an exon, and an unpaired splice donor site by homologous recombination, such that a homologously recombinant cell comprising a new transcription unit is formed. The new transcription unit can be used to turn the gene on or off as desired. This method of affecting endogenous gene expression is taught in U.S. Pat. No. 5,641,670.

Optionally, expression of a polypeptide of the invention can be altered in cells which have been removed from a mammal, such as dermal cells. The cells can then be replaced into the same or another mammal, to or within the vicinity of a region which hair follicle formation is to be modulated.

In another embodiment, recombinantly-produced or chemically-synthesized polypeptide of the invention can be used to increase said polypeptide levels in a cell where it is desirable to increase or stimulate inner root sheath development or hair follicle formation. The polypeptides can be used in a pharmaceutically acceptable composition and can be applied topically, as is well-known in the art and described herein.

In general, recombinant production of a polypeptide of the invention requires incorporation of nucleic acid sequences encoding a polypeptide of the invention (e.g., GATA-3, SEQ ID NO:1; BMPR1A, SEQ ID NO:2) into a recombinant expression vector in a form suitable for expression of the protein in a host cell.

A suitable form for expression provides that the recombinant expression vector, viral vector, or plasmid includes one or more regulatory sequences operatively-linked to the nucleic acids encoding the polypeptide of the invention in a manner which allows for transcription of the nucleic acids into mRNA and translation of the mRNA into the protein. Regulatory sequences can include promoters, enhancers and other expression control elements (e.g., polyadenylation signals). Such regulatory sequences are known to those skilled in the art and are described in Goeddel D. D., ed., Gene Expression Technology, Academic Press, San Diego, Calif. (1991). It should be understood that the design of the vector disclosed herein can depend on such factors as the choice of the host cell to be transfected and/or the level of expression required. Nucleic acid sequences or vectors harboring nucleic acid sequences encoding a polypeptide of the invention can be introduced into a host cell, which can be of eukaryotic or prokaryotic origin, by standard techniques for transforming cells. Suitable methods for transforming host cells can be found in Sambrook, et al. (Molecular Cloning: A Laboratory Manual, 3rd Edition, Cold Spring Harbor Laboratory Press (2000)) and other laboratory manuals. The number of host cells transformed with a nucleic acid sequence encoding a polypeptide of the invention will depend, at least in part, upon the type of expression vector used and the type of transformation technique used. Nucleic acids can be introduced into a host cell transiently, or more typically, for long-term expression of a polypeptide of the invention, the nucleic acid sequence is stably integrated into the genome of the host cell or remains as a stable episome in the host cell. Once produced, a polypeptide of the invention can be recovered from culture medium as a secreted polypeptide, although it also may be recovered from host cell lysates when directly expressed without a secretory signal. When a polypeptide of the invention is expressed in a recombinant cell other than one of human origin, the polypeptide is substantially free of proteins or polypeptides of human origin. However, it may be necessary to purify the polypeptide of the invention from recombinant cell proteins using conventional protein purification methods to obtain preparations that are substantially homogeneous as to said polypeptide. As a first step, the culture medium or lysate is centrifuged to remove particulate cell debris. The membrane and soluble protein fractions are then separated. The recombinant protein may then be purified from the soluble protein fraction. The recombinant protein thereafter is purified from contaminant soluble proteins and polypeptides using any of the following suitable purification procedures: by fractionation on immunoaffinity or ion-exchange columns; ethanol precipitation; reverse phase HPLC; chromatography on silica or on a cation-exchange resin such as DEAE; chromatofocusing; SDS-PAGE; ammonium sulfate precipitation; gel filtration using, for example, SEPHADEX G-75; ligand affinity chromatography, and protein A SEPHAROSE columns to remove contaminants such as IgG.

In addition to recombinant production, a polypeptide of the invention can be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431A Peptide Synthesizer (Perkin Elmer, Boston, Mass.). Various fragments of the polypeptide can be chemically-synthesized separately and combined using chemical methods to produce a full-length molecule.

Whether recombinantly-produced or chemically-synthesized, a polypeptide of the invention or portion thereof can be further modified for use. For example, isolated polypeptide can be glycosylated or phosphorylated using well-known methods prior to its use in promoting or stimulating inner root sheath development or hair follicle formation.

In a further embodiment of the present invention, the expression or activity of a polypeptide of the invention can be regulated using an agent which alters the expression of nucleic acid sequences encoding said polypeptide or an agent which alters protein activity. Agents suitable for regulating the expression or activity of polypeptide of the invention encompass numerous chemical classes, though typically they are organic molecules or small organic compounds having a molecular weight of more than 100 and less than about 2,500 daltons. Agents comprise functional groups necessary for structural interaction with proteins, particularly hydrogen bonding, and typically include at least an amine, carbonyl, hydroxyl or carboxyl group, preferably at least two of the functional chemical groups. The agents often comprise cyclical carbon or heterocyclic structures and/or aromatic or polyaromatic structures substituted with one or more of the above functional groups. Agents can also be found among biomolecules including peptides, antibodies, saccharides, fatty acids, steroids, purines, pyrimidines, derivatives, nucleic acid sequences such as RNA molecules provided herein and structural analogs or combinations thereof.

Agents are obtained from a wide variety of sources including libraries of synthetic or natural compounds. The use of replicable genetic packages, such as the bacteriophages, is one method of generating novel polypeptide entities that regulate the expression or activity of a polypeptide of the invention. This method generally consists of introducing a novel, exogenous DNA segment into the genome of a bacteriophage (or other amplifiable genetic package) so that the polypeptide encoded by the non-native DNA appears on the surface of the phage. When the inserted DNA contains sequence diversity, then each recipient phage displays one variant of the template amino acid sequence encoded by the DNA, and the phage population (library) displays a vast number of different but related amino acid sequences.

Antibodies which specifically bind a polypeptide of the invention are also contemplated as antagonistic or agonist agents for regulating the activity of a polypeptide of the invention. Antibodies to a polypeptide of the invention can be generated using methods that are well-known in the art. Such antibodies can include, but are not limited to, polyclonal, monoclonal, chimeric, single chain, Fab fragments, and fragments produced by a Fab expression library. Neutralizing antibodies are especially preferred for therapeutic use.

For the production of antibodies, various hosts including goats, rabbits, rats, mice, humans, and others, can be immunized by injection with polypeptide of the invention or any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface-active substances such as lysolecithin, pluronic polyols, polyanions, peptides, oil emulsions, keyhole limpet hemocyanin, and dinitrophenol. Among adjuvants used in humans, BCG (bacilli Calmette-Guerin) and Corynebacterium parvum are especially preferable.

When oligopeptides, peptides, or fragments are used to induce antibodies to a polypeptide of the invention they usually consist of at least five amino acids or at least 10 amino acids. In general these peptides are identical to a portion of the amino acid sequence of the natural protein, and may contain the entire amino acid sequence of a small, naturally occurring molecule. Short stretches of a polypeptide of the invention can be fused with those of another protein such as keyhole limpet hemocyanin and antibody produced against the chimeric molecule.

Monoclonal antibodies to a polypeptide of the invention can be prepared using any technique which provides for the production of antibody molecules by continuous cell lines in culture. These include, but are not limited to, the hybridoma technique, the human B-cell hybridoma technique, and the EBV-hybridoma technique (Kohler, et al. (1975) Nature 256:495-497; Kozbor, et al. (1985) J. Immunol. Methods 81:31-42; Cote, et al. (1983) Proc. Natl. Acad. Sci. 80:2026-2030; Cole, et al. (1984) Mol. Cell Biol. 62:109-120).

In addition, techniques developed for the production of humanized and chimeric antibodies, the splicing of mouse antibody genes to human antibody genes to obtain a molecule with appropriate antigen specificity and biological activity can be used (Morrison, et al. (1984) Proc. Natl. Acad. Sci. 81, 6851-6855; Neuberger, et al. (1984) Nature 312:604-608; Takeda, et al. (1985) Nature 314:452-454). Alternatively, techniques described for the production of single chain antibodies can be adapted, using methods known in the art, to produce a single chain antibody which is specific to a polypeptide of the invention. Antibodies with related specificity, but of distinct idiotypic composition, can be generated by chain shuffling from random combinatorial immunoglobulin libraries (Burton (1991) Proc. Natl. Acad. Sci. 88, 11120-11123).

Antibodies can also be produced by inducing in vivo production in the lymphocyte population or by screening immunoglobulin libraries or panels of highly specific binding reagents as is well-known in the art (Orlandi, et al. (1989) Proc. Natl. Acad. Sci. 86: 3833-3837; Winter, et al. (1991) Nature 349:293-299).

Antibody fragments, which contain specific binding sites for a polypeptide of the invention, can also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries can be constructed to allow rapid and easy identification of monoclonal Fab fragments with the desired specificity (Huse, et al. (1989) Science 254:1275-1281).

Various immunoassays can be used for screening to identify antibodies having the desired specificity. Numerous protocols for competitive binding or immunoradiometric assays using either polyclonal or monoclonal antibodies with established specificity are well-known in the art. Such immunoassays typically involve the measurement of complex formation between a specific antibody and a polypeptide of the invention. A two-site, monoclonal-based immunoassay utilizing monoclonal antibodies reactive to two non-interfering epitopes can be used or alternatively a competitive binding assay can be employed.

As will be appreciated by one of skill in the art, a full-length polypeptide of the invention can be produced for use in the methods of the invention, however, fragments of a polypeptide of the invention can also be used provided the fragment maintains the desired binding interaction or activity of the full-length protein. It is also contemplated that it may be desirable to produce a polypeptide of the invention which maintains specific binding sites but lacks one or more other activities.

Isolated polypeptides, constructs, RNA molecules, or antibodies can be used for regulating the expression or activity of polypeptide of the invention so that epithelial stem cell lineages are modulated and to identify these and other agents for this purpose, the present invention provides screening assays.

Accordingly, the present invention provides a method for identifying agents which interact with the E-cadherin gene or with the components which regulate the expression of products of nucleic acid sequences encoding E-cadherin to modulate epithelial stem cell lineage. For example, the agent can interact with a Wnt, a BMP inhibitor, Lef1, or β-catenin, to affect a change in their expression or stability so that E-cadherin expression is either increased or decreased. In one embodiment, agents will decrease, interfere with or inhibit the expression of products of nucleic acid sequences encoding E-cadherin so that epithelial bud formation is stimulated. Examples of such agents include, but are not limited to, antisense molecules, RNAi or ribozymes targeted to the E-cadherin gene which inhibit expression of products of nucleic acid sequences encoding E-cadherin; means for introduction of mutations into the E-cadherin gene which inhibit expression of products of nucleic acid sequences encoding E-cadherin or produce a E-cadherin polypeptide with decreased stability; and small organic molecules or peptides which are capable of inhibiting expression of products of nucleic acid sequences encoding E-cadherin themselves (e.g., by binding to the promoter region of the gene to inhibit transcription and subsequent expression); small organic molecules or peptides which are capable of increasing expression of products of nucleic acid sequences encoding a Wnt or Lef1 or stabilizing β-catenin.

In an alternative embodiment, agents will increase, activate or stimulate expression of products of nucleic acid sequences encoding E-cadherin so that epithelial bud formation is reduced. Examples of such agents include, but are not limited to, means for introduction of mutations into the E-cadherin gene which stimulate expression of products of nucleic acid sequences encoding E-cadherin; small organic molecules or peptides which are capable of increasing or stimulating expression of products of nucleic acid sequences encoding E-cadherin themselves (e.g., by binding to the promoter region of the gene to promote transcription and subsequent expression); antisense molecules, RNAi or ribozymes targeted to the Lef1, a Wnt, or β-catenin gene which inhibit expression of products of nucleic acid sequences encoding Lef1, a Wnt, or β-catenin, respectively; means for introduction of mutations into the Lef1, a Wnt, or β-catenin gene which inhibit expression of products of nucleic acid sequences encoding Lef1, a Wnt, or β-catenin or produce a Lef1, a Wnt, or β-catenin polypeptide with decreased stability; and small organic molecules or peptides which are capable of inhibiting expression of products of nucleic acid sequences encoding Lef1, a Wnt, or β-catenin themselves (e.g., by binding to the promoter region of the gene to promote transcription and subsequent expression).

This method of the invention involves contacting a test cell, which contains a reporter gene (e.g., GFP) operably linked to an E-cadherin promoter, with an agent and then detecting the expression of products of nucleic acid sequences encoding the reporter in the test cell. An agent which causes an increase or decrease in expression of products of nucleic acid sequences encoding the reporter in the test cell when compared a test cell not contacted with the agent, indicates that the agent modulates epithelial stem cell lineage in the test cell. Test cells expressing a product of nucleic acids encoding a reporter which can be used in accordance with this method of the invention include, but are not limited to, keratinocytes, cancer cells, or epithelial cells (e.g., MDCK cells) containing nucleic acid sequences encoding a Wnt, a BMP inhibitor, Lef1 and β-catenin which can be generated in accordance with methods disclosed herein, or other methods well-known in the art. Various E-cadherin promoter sequences can be generated by PCR using DNA from the E-cadherin genomic locus as template. Nucleic acid sequences corresponding to the E-cadherin genomic locus are readily available from databases such as GENEBANK and EMBL. Primers can be synthesized corresponding to the 5′ and 3′ boundaries of the selected promoter regions. Primers also can contain additional restriction enzyme recognition sequences to facilitate subcloning.

This method for screening for agents that modulate epithelial stem cell lineage involves culturing a test cell which contains nucleic acid sequences encoding a Wnt, a BMP inhibitor, Lef1, β-catenin and a reporter, such as GFP for illustrative purposes, operably linked to the E-cadherin promoter; adding at least one test agent to a point of application, such as a well, in the plate and incubating the plate for a time sufficient to allow the test agent to effect GFP accumulation; detecting fluorescence of the test cells contacted with the test agent, wherein fluorescence indicates expression of the GFP polypeptide in the test cells; and comparing the fluorescence of test cells not contacted with the test agent. A decrease in fluorescence of the test cell contacting the test agent relative to the fluorescence of test cells not contacting the test agent indicates that the test agent causes a decrease in expression of products of nucleic acid sequences encoding E-cadherin in the test cell and an increase in epithelial bud formation. An increase in fluorescence of the test cell contacting the test agent relative to the fluorescence of test cells not contacting the test agent indicates that the test agent causes an increase in expression of products of nucleic acid sequences encoding E-cadherin in the test cell and a reduction in epithelial bud formation.

It is contemplated that more than one test agent can be tested in a single point of application to effect a change in the expression or stability of more than one nucleic acid sequence or protein involved in epithelial stem cell lineage.

In one embodiment, the test cell of the screening method of the invention has an elevated level of stabilized β-catenin. Such cells are useful in identifying agents that induce Lef1. In an alternative embodiment, the test cell has an elevated level of stable Lef1. Such cells are useful in identifying agents that induce stabilized β-catenin. Elevated levels of β-catenin or Lef1 can be accomplished by the addition of exogenous agents known to increase the expression or stability of said proteins or by recombinant protein expression wherein said proteins are expressed from a constitutive promoter. In another embodiment of the screening method of the invention, the test cell is treated with a Wnt and a Bmp inhibitor prior to the addition of the test agent to identify agents which elevate E-cadherin promoter activity. Such agents would be useful as inhibitors of epithelial bud formation.

Another embodiment of the present invention is a method for identifying an agent which modulates epithelial stem cell lineages by contacting a test cell, which contains a nucleic acid sequence encoding a reporter operably linked to a GATA-3 promoter or a promoter containing a GATA-3 binding site, with an agent and detecting expression of a product of the nucleic acid sequence encoding the reporter in the test cell. An agent which causes an increase or decrease in expression of products of nucleic acid sequences encoding the reporter in the test cell when compared a test cell not contacted with the agent, indicates that the agent increases or decreases, respectively, GATA-3 expression thereby modulating epithelial stem cell lineages.

Agents for increasing or decreasing the expression of GATA-3 protein can be identified by operably linking a GATA-3 promoter (e.g., SEQ ID NO:3) to a reporter. In this assay, activators or repressors of GATA-3 expression can include agents which directly bind to a region on the promoter as well as agents which interact with transcriptional factors that bind to the GATA-3 promoter thereby specifically regulating the expression of GATA-3. The steps involved in this screening assay include, culturing a test cell which contains nucleic acid sequences encoding a reporter operably linked to a GATA-3 promoter; adding at least one test agent to a point of application, such as a well, in the plate containing the test cell and incubating the plate for a time sufficient to allow the test agent to effect reporter accumulation; detecting reporter activity of the test cell contacted with the test agent, wherein reporter activity indicates expression of the reporter polypeptide in the test cell; and comparing reporter activity of the test cell which has been contacted with the test agent to that of the test cell not contacted with the test agent. A decrease in reporter activity of the test cell contacting the test agent relative to the reporter activity of the test cell not contacting the test agent indicates that the test agent causes a decrease in expression of products of nucleic acid sequences encoding GATA-3 in the test cell. An increase in reporter activity of the test cell contacting the test agent relative to the reporter activity of the test cell not contacting the test agent indicates that the test agent causes an increase in expression of products of nucleic acid sequences encoding GATA-3 in the test cell.

In other embodiments, the transcriptional regulatory activity of GATA-3 is assayed by linking a reporter to one or more GATA-3 binding sites fused to other suitable promoter sequences. When assaying for regulators of GATA-3 activity using a promoter containing GATA-3 binding sites, test cells can contain endogenous GATA-3 protein or can be engineered to express GATA-3 protein.

Agents which increase or stimulate the activator activity of GATA-3 can be identified by operably linking to a reporter, one or more GATA-3 binding sites located upstream of a minimal promoter. In general, at least one GATA-3 binding site is upstream of the minimal promoter. Alternatively, two or three GATA-3 binding sites are upstream of the minimal promoter. Optionally, five to ten GATA-3 binding sites are upstream of the minimal promoter. In this assay, the promoter is silent until GATA-3 is activated or present. This assay can be conducted by adding at least one test agent to a test cell containing the above described reporter gene construct and incubating the test cell for a time sufficient to allow the test agent to effect reporter accumulation via GATA-3 activity. Subsequently, reporter activity of the test cell contacted with the test agent is detected and compared with reporter activity of test cell not contacted with the test agent. An increase in reporter activity of the test cell contacting the test agent relative to the reporter activity of the test cell not contacting the test agent indicates that the test agent causes an increase in expression or activity of GATA-3 in the test cell. To differentiate whether the agent is regulating expression or activity of GATA-3, the agent can be further screened on a test cell containing a nucleic acid sequence encoding a reporter operably linked to a GATA-3 promoter. If the agent does not regulate the expression of the GATA-3 promoter in this test cell, it can be concluded that it regulates the activator activity of GATA-3.

Agents for increasing or stimulating the repressor activity of GATA-3 can be identified by operably linking to a reporter, A K14 promoter and enhancer with one or more GATA-3 binding sites located between the K14 promoter and enhancer. A suitable K14 promoter and enhancer is described in Vassar, et al. ((1989) Proc. Natl. Acad. Sci. USA. 86(5):1563-7). In one embodiment, the 5′-to-3′ orientation of the K14 promoter, enhancer, and GATA-3 binding sites is K14 enhancer->GATA-3 binding sites->K14 promoter. Alternatively, an enhancer can be located in an intron or 3′ of the reporter. In this assay, the promoter would be active, for example in keratinocytes, until GATA-3 is activated or present. This assay can be conducted by adding at least one test agent to a test cell containing the above described reporter gene construct and incubating the test cell for a time sufficient to allow the test agent to effect reporter accumulation via GATA-3 activity. Subsequently, reporter activity of the test cell contacted with the test agent is detected and compared with reporter activity of test cell not contacted with the test agent. A decrease in reporter activity of the test cell contacting the test agent relative to the reporter activity of the test cell not contacting the test agent indicates that the test agent causes an increase in expression or repressor activity of GATA-3 in the test cell. To differentiate whether the agent is regulating expression or activity of GATA-3, the agent may be further screened on a test cell containing a nucleic acid sequence encoding a reporter operably linked to a GATA-3 promoter. If the agent does not regulate the expression of the GATA-3 promoter in this test cell, it can be concluded that it regulates the repressor activity of GATA-3.

A still further embodiment of the present invention relates to a screening assay for the identification of an agent which modulates BMPR1A expression. In general, such a method involves contacting a test cell, which contains a nucleic acid sequence encoding a reporter operably linked to a BMPR1A promoter, with an agent and detecting expression of a product of the nucleic acid sequence encoding the reporter in the test cell. An agent which causes an increase or decrease in expression of a product of a nucleic acid sequence encoding the reporter in the test cell when compared a test cell not contacted with the agent, indicates that the agent increases or decreases, respectively, BMPR1A expression thereby modulating inner root sheath and/or hair shaft development or formation. Test cells which can be used in accordance with this screening assay of the invention include, but are not limited to, keratinocytes, cancer cells, or epithelial cells (e.g., MDCK cells), which contain a nucleic acid sequence encoding a reporter operably linked to a selected promoter of interest, in this case a BMPR1A promoter. Such test cells can also be of a transgenic animal.

In this screening assay of the invention, activators or repressors of BMPR1A expression can include agents which directly bind to a region on the promoter as well as agents which interact with transcriptional factors that bind to the BMPR1A promoter thereby specifically regulating the expression of BMPR1A. The steps involved in this screening assay include, culturing a test cell which contains nucleic acid sequences encoding a reporter operably linked to a BMPR1A promoter; adding at least one test agent to a point of application, such as a well, in the plate containing the test cell and incubating the plate for a time sufficient to allow the test agent to effect reporter accumulation; detecting reporter activity of the test cell contacted with the test agent, wherein reporter activity indicates expression of the reporter polypeptide in the test cell; and comparing reporter activity of the test cell which has been contacted with the test agent to that of the test cell not contacted with the test agent. A decrease in reporter activity of the test cell contacting the test agent relative to the reporter activity of the test cell not contacting the test agent indicates that the test agent causes a decrease in expression of products of nucleic acid sequences encoding BMPR1A in the test cell. An increase in reporter activity of the test cell contacting the test agent relative to the reporter activity of the test cell not contacting the test agent indicates that the test agent causes an increase in expression of products of nucleic acid sequences encoding BMPR1A in the test cell.

In a second screening assay for identifying agents which modulate BMPR1A activity, the assay is carried out at the level of pathway activation so that inner root sheath and/or hair shaft formation is stimulated or inhibited. This method involves contacting a test cell, such as a keratinocyte, which contains (BMPR1A+) or lacks a functional BMPR1A (BMPR1A−), with an agent and detecting the phosphorylation state of Smad-1 in the test cell. The phosphorylation state of Smad-1 is indicative of the activity of the BMPR1A signaling pathway. Upon ligand engagement of BMPR1A, Smad-1 is phosphorylated and activated. Thus, if Smad-1 becomes phosphorylated in a BMPR1A− cell upon exposure to a test agent, said test agent is said to activate a BMPR1A pathway. Conversely, if phosphorylation of Smad-1 is blocked in a BMPR1A+ cell upon exposure to a test agent, said test agent is said to inhibit a BMPR1A pathway. Methods of detecting the phosphorylation state of a protein are well-known in the art, however, it is desirable that antibodies which specifically recognize phosphorylated or unphosphorylated forms of Smad-1 be used in accordance with the method of the invention.

In a third screening assay for identifying agents which modulate hair shaft development, a test cell, which lacks functional BMPR1A and contains a nucleic acid sequence encoding a reporter operably linked to a Wnt-responsive promoter, is contacted with a test agent and expression of a product of the nucleic acid sequence encoding the reporter in the test cell is detected.

Cells lacking functional BMPR1A include cells which lack expression of BMPR1A (i.e., a Bmpr1a null cell) or which have a mutated BMPR1A protein which is not active (e.g., does not bind a BMP or is blocked in its ability to transmit a BMP signal). Methods of generating cells lacking functional BMPR1A are described herein.

A Wnt-responsive promoter for use in this method of the invention can include a promoter exemplified herein (i.e., TOP and HK1), as well as a promoter or Wnt-responsive promoter element from the following loci: Lef1, hair keratin, Foxn1, engrailed-2, krox-20, XA-1, xCRISP, UVS.2, UVS.2-related genes, xONR1, connexin 30, or retinoic acid receptor gamma (McGrew et al. (1999) Mech. Dev. 87(1-2):21-32). Further, a mutated promoter such as FOPFlash can be used as a control to determine whether an agent is specifically increasing the expression of a nucleic acid sequence operably linked to a Wnt-responsive promoter. Wnt-responsive promoter-reporter constructs are well-known in the art (e.g., TOPFlash and FOPFlash are commercially available from Upstate Biotechnology, Lake Placid, N.Y.).

This method can be conducted by adding at least one test agent to a test cell lacking functional BMPR1A and containing the Wnt-responsive promoter-reporter gene construct and incubating the test cell for a time sufficient to allow the test agent to effect reporter accumulation in the absence of BMPR1A activity. Subsequently, reporter activity of the test cell contacted with the test agent is detected and compared with reporter activity of test cell not contacted with the test agent. An increase in reporter activity of the test cell contacting the test agent relative to the reporter activity of the test cell not contacting the test agent indicates that the test agent causes an increase in expression of a Wnt-responsive gene in the test cell. It is contemplated that, in this assay, an agent which increases the expression of a nucleic acid sequence operably linked to a Wnt-responsive promoter is able to bypass or overcome the deficiency in BMPR1A activity and will thus be useful in increasing hair shaft formation. An agent screened in accordance with this assay of the invention can include any of the agents disclosed herein.

In assays utilizing a reporter, the reporter gene sequence(s) is inserted into a recombinant expression vector. A reporter gene construct refers to a plasmid, virus or other vehicle known in the art that has been manipulated by insertion or incorporation of nucleic acid sequences encoding a reporter. Such reporter gene constructs of the invention are typically plasmids which contain at least a portion of an E-cadherin, GATA-3 or BMPR1A promoter sequence which is operably associated with the inserted nucleic acid sequences encoding the reporter. The construct typically contains an origin of replication as well as specific selectable or screenable marker genes for initially isolating, identifying or tracking test cells that contain the desired reporter/promoter DNA. The reporter gene construct also can provide unique or conveniently located restriction sites to allow severing and/or rearranging portions of the DNA inserts in a reporter gene construct. More than one reporter gene can be inserted into the construct such that the test cells containing the resulting construct can be assayed by different means.

A promoter which is operably associated or operably linked to nucleic acid sequences encoding a reporter means that the sequences are joined and positioned in such a way as to permit transcription. Two or more sequences, such as a promoter and any other nucleic acid sequences are operably associated if transcription commencing in the promoter will produce an RNA transcript of the operably associated sequences.

When screening for agents which regulate the expression of BMPR1A, GATA-3 or E-cadherin, a reporter should be operably linked to a said promoter. A promoter is defined as a fully functional promoter which regulates expression of its corresponding messenger RNA, i.e., a promoter which has binding sites for transcription factors and minimal promoter sequences. An exemplary BMPR1A promoter can include sequences upstream of the BMPR1A coding region of accession number NT_{—}030059.

A GATA-3 promoter typically has sequences necessary for GATA-3 self-regulation as well as binding sites for other transcription factors and minimal promoter sequences. Exemplary GATA-3 promoters include, but are not limited to, accession number X73519 (SEQ ID NO:3) and the promoter taught by Lakshmanan, et al. ((1999) Mol. Cell. Biol. 19:1558-1568). Alternatively, a promoter containing a GATA-3 binding site can be used and is defined as a promoter which contains minimal GATA-3 binding sites, e.g., AGATCTTA (SEQ ID NO:4) (Ko and Engel (1993) Mol. Cell. Biol. 13(7):4011-22) in addition to minimal regulatory sequences and/or sequences from other suitable promoters. A GATA-3 promoter/reporter construct can also contain the 5′ or 3′ untranslated regions (see, e.g., accession number AJ131811; SEQ ID NO:5) or introns of GATA-3 to which regulatory proteins can bind (see, e.g., Hwang, et al. (2002) J. Immunol. 169(1):248-53).

Various portions of an E-cadherin, GATA-3, or BMPR1A promoter or gene sequence can be generated by PCR or other conventional cloning techniques using DNA from the respective genomic locus. For PCR amplification, primers can be synthesized corresponding to the 5′ and 3′ boundaries of the selected promoter or gene regions. Primers also can contain additional restriction enzyme recognition sequences to facilitate subcloning.

A reporter gene refers to any sequence that is detectable and distinguishable from other genetic sequences present in test cells. Preferably, the reporter nucleic acid sequence encodes a protein that is readily detectable either by its presence, or by its activity that results in the generation of a detectable signal. A nucleic acid sequences encoding the reporter are used in the invention to monitor and report the activity of an E-cadherin, GATA-3, or BMPR1A promoter in test cells.

A variety of enzymes can be used as reporters including, but are not limited to, β-galactosidase (Nolan, et al. (1988) Proc. Natl. Acad. Sci. USA 85:2603-2607), chloramphenicol acetyltransferase (CAT; Gorman, et al. (1982) Mol. Cell Biol. 2:1044; Prost, et al. (1986) Gene 45:107-111), β-lactamase, β-glucuronidase and alkaline phosphatase (Berger, et al. (1988) Gene 66:1-10; Cullen, et al. (1992) Meth. Enzymol. 216:362-368). Transcription of the nucleic acid sequences encoding a reporter leads to production of the enzyme in test cells. The amount of enzyme present can be measured via its enzymatic action on a substrate resulting in the formation of a detectable reaction product. The method of the invention provides means for determining the amount of reaction product, wherein the amount of reaction product generated or the remaining amount of substrate is related to the amount of enzyme activity. For some enzymes, such as β-galactosidase, β-glucuronidase and β-lactamase, well-known fluorogenic substrates are available that allow the enzyme to convert such substrates into detectable fluorescent products.

A variety of bioluminescent, chemiluminescent and fluorescent proteins also may be used as light-emitting reporters. Exemplary light-emitting reporters, which are enzymes and require cofactor(s) to emit light, include, but are not limited to, the bacterial luciferase (luxAB gene product) of Vibrio harveyi (Karp (1989) Biochim. Biophys. Acta 1007:84-90; Stewart, et al. (1992) J. Gen. Microbiol. 138:1289-1300), and the luciferase from firefly, Photinus pyralis (De Wet, et al. (1987) Mol. Cell. Biol. 7:725-737).

Another type of light-emitting reporter, which does not require substrates or cofactors includes, but is not limited to, the wild-type green fluorescent protein (GFP) of Victoria aequoria (Chalfie, et al. (1994) Science 263:802-805), modified GFPs (Heim, et al. (1995) Nature 373:663-4; WO 96/23810), and the gene products encoded by the Photorhabdus luminescens lux operon (luxABCDE) (Francis, et al. (2000) Infect. Immun. 68(6):3594-600). Transcription and translation of these types of reporters leads to the accumulation of the fluorescent or bioluminescent proteins in test cells, which can be measured by a device, such as a fluorimeter, flow cytometer, or luminometer. Methods for performing assays on fluorescent materials are well-known in the art (e.g., Lackowicz (1983) In: Principles of Fluorescence Spectroscopy, New York, Plenum Press).

For convenience and efficiency, enzymatic reporters and light-emitting reporters are used for the screening assays of the invention. Accordingly, the invention encompasses histochemical, colorimetric and fluorometric assays. An exemplary reporter construct, provided herein, contains the E-cadherin promoter which regulates the transcription and translation (expression) of the reporter, GFP. To measure a decrease, a destabilized form of GFF (dsGFP) with a shortened half-like is used.

Introduction of the reporter gene construct into the test cells can be carried out by conventional techniques well-known to those skilled in the art, such as transformation, conjugation, and transduction.

In addition to conventional chemical methods of transformation, reporter gene constructs of the invention can be introduced into a test cell by physical means, such as by electroporation or microinjection. Electroporation allows transfer of the vector by high voltage electric impulse, which creates pores in the plasma membrane of the cell and is performed according to methods well-known in the art. Additionally, the reporter gene construct can be introduced into test cells by protoplast fusion, using methods well-known in the art. The reporter gene construct can be introduced into a test cell transiently, or more typically, the nucleic acids are stably integrated into the genome of the test cell or remain as stable episomes in the test cell.

The test cells which contain the nucleic acid sequences encoding the reporter and which express products of the nucleic acid sequences encoding the reporter can be identified by at least four general approaches; detecting DNA-DNA or DNA-RNA hybridization; observing the presence or absence of marker gene functions (e.g., resistance to antibiotics); assessing the level of transcription as measured by the expression of reporter mRNA transcripts in the host cell; and detecting the reporter gene product as measured by immunoassay or by its biological activity.

The test cells can be cultured under standard conditions of temperature, incubation time, optical density, plating density and media composition corresponding to the nutritional and physiological requirements of the cells. However, conditions for maintenance and growth of the test cell can be different from those for assaying candidate test compounds in the screening methods of the invention. Modified culture conditions and media are used to facilitate detection of the expression of a reporter molecule. Any techniques known in the art may be applied to establish the optimal conditions.

Screening assays of the invention can be performed in any format that allows rapid preparation and processing of multiple reactions such as in, for example, multi-well plates of the 96-well variety. Stock solutions of the agents as well as assay components are prepared manually and all subsequent pipeting, diluting, mixing, washing, incubating, sample readout and data collecting is done using commercially available robotic pipeting equipment, automated work stations, and analytical instruments for detecting the signal generated by the assay.

In addition to the test agent and test cell, a variety of other reagents can be included in the screening assays. These include reagents like salts, neutral proteins, e.g., albumin, detergents, etc. Also, reagents that otherwise improve the efficiency of the assay, such as protease inhibitors, nuclease inhibitors, anti-microbial agents, and the like can be used.

Agents identified in the screening assays provided herein are useful in modulating epithelial stem cell lineages or for stimulating or inhibiting inner root sheath development or hair follicle formation.

Accordingly, purified polypeptides of the invention, constructs, RNA molecules or agents which modulate the expression or activity of a polypeptide of the invention can be formulated into pharmaceutical compositions comprising an effective amount of the active compound and a pharmaceutically acceptable vehicle. Such pharmaceutical compositions can be prepared by methods and contain vehicles which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000. For example, sterile saline and phosphate-buffered saline at physiological pH may be used. Preservatives, stabilizers, dyes and even flavoring agents can be provided in the pharmaceutical composition. For example, sodium benzoate, sorbic acid and esters of p-hydroxybenzoic acid can be added as preservatives. In addition, antioxidants and suspending agents can be used. Liposomes, such as those described in U.S. Pat. No. 5,422,120, WO 95/13796, WO 91/14445, or EP 524,968 B1, may also be used as a carrier.

By effective amount it is meant an amount of active compound which inhibits or stimulates hair follicle formation. A pharmaceutical composition can be administered to a cell (e.g., a progenitor cell, a dermal papilla, a matrix cell or fibroblast to then act on the skin epithelium) or a host, preferably a human, to inhibit or stimulate hair follicle formation in said host.

A pharmaceutical composition of the invention can be administered by any suitable means, including parenteral injection (such as intraperitoneal, subcutaneous, or intramuscular injection), orally, or by topical application (e.g., transdermal or via a mucosal surface). Preferably, a pharmaceutical composition of the invention is for topical administration in the form of a cream, lotion, liquid, ointment, gel, or aerosol.

Agents identified in the screening assays provided herein are useful in stimulating or inhibiting epithelial bud formation to modulate the development of hair follicles, teeth, lungs and the like.

The invention is described in greater detail by the following non-limiting examples.

### Example 1

### Materials and Methods

Mice. The generation and characterization of the Bmpr1a fl/fl mice is known in the art (Mishina et al. (2002) supra). Bmpr1a mice were mated with established K14-Cre mice (Vasioukhin et al. (1999) supra) to generate mice homozygous for the loss of BMPR1A function in skin epithelium. K14-Cre is active by E9 of skin development, and is effective at quantitative ablation by E15. These mice were also mated on the background of TOPGAL transgenic mice, driving expression of β-galactosidase only under conditions where cells are responsive to Wnt signaling and already express a member of the Lef1/Tcf family of DNA binding proteins (DasGupta and Fuchs (1999) supra).

GATA-3nlslacZ mice are known in the art (Hendriks, et al. (1999) supra; van Doorninck, et al. (1999) supra). Briefly, the lacZ gene fused to a nuclear localization signal (nls) was placed in-frame at the ATG translational start site in the GATA-3 locus, inactivating the GATA-3 gene and expressing β-galactosidase from the endogenous GATA-3 promoter. To obtain GATA-3 null embryos that survive to E18.5, a modification of a drug rescue regime was used (Lim, et al. (2000) supra). Beginning at E7.5, pregnant GATA-3nlslacZ/+dams which were mated to GATA-3nlslacZ/+males were given fresh water daily containing 100 μg/mL of L-phenylephrine (SIGMA, St. Louis, Mo.), 100 μg/mL of isoproterenol (SIGMA, St. Louis, Mo.) and 2 mg/mL of ascorbic acid (SIGMA, St. Louis, Mo.). The presence of the GATA-3nlslacZ allele was determined by a combination of PCR screening, X-gal staining, phenotypic identification of GATA-3 null embryos, and/or loss of GATA-3 immunoreactivity (Lim, et al. (2000) supra). PCR primers used were 5′-TCC TGC GAG CCT GGC TGT CGG A-3′ (SEQ ID NO:6) which recognizes GATA-3 intron 1,5′-CCT GTA GCC AGC TTT CAT AAC-3′ (SEQ ID NO:7) which recognizes lacZ, and 5′-GTT GCC TTG ACC ATC GAT GTT-3′ (SEQ ID NO:8) which recognizes GATA-3 exon 2. Reaction conditions were 94° C. for 5 minutes, followed by 45 cycles of 94° C. for 30 seconds, 53° C. for 30 seconds and 72° C. for 1.2 minutes. Band sizes were: ˜750 bp (wild-type allele) and ˜1.1 kb (GATA-3nlslacz allele).

Noggin and Shh null mice, and Lef1 null mice are well-known in the art. β-Catenin conditional null, TOPGAL, K14-Lef1 and K14ΔNβcat animals have been described (Zhou, et al. (1995) supra; Gat, et al. (1998) supra; DasGupta and Fuchs (1999) supra; Vasioukhin, et al. (2001) supra). K14-EcadherinHA transgenic mice were generated using standard methods.

Plasmid Construction. The E-cadherin promoter was generated by PCR with primers (Celera Genomics database) and a BAC clone as template. Mutation of the Lef1 binding site (−242 to −233) was achieved using the QUICKCHANGE® Site-Directed Mutagenesis Kit (Stratagene, La Jolla, Calif.). Promoter fragments were subcloned into pGL3-basic (luciferase; Promega, Madison, Wis.) or pNASSβ(M) (β-galactosidase; DasGupta and Fuchs (1999) supra). HA-tagged Snail cDNA was subcloned into the BamHI/NotI sites of the K14-cassette (Gat, et al. (1998) supra). E-cadherin-HA cDNA was generated by PCR amplification of pKS+UM plasmid with a forward primer at the XhoI site (3′-end), and a reverse primer containing the HA-tag, a stop codon and XbaI site. Ecadherin-HA localized to intercellular junctions in keratinocytes.

Nuclear Lef1 and β-catenin Generated by Noggin- and Wnt3a-Conditioned Media. Keratinocytes from newborn mouse skin were cultured in low calcium E-media (DasGupta and Fuchs (1999) supra), and then either treated for a) 12 hours with control-media (nog−) or conditioned-media (nog+) from a noggin-secreting cell line, or b) 5 hours with control-media (wnt−) or conditioned-media (wnt+) from a Wnt3A-secreting cell line. In some cases, 7 hour noggin/control-media was replaced with noggin/wnt-conditioned or nog(−)/Wnt(−)-media for 5 hours. In all cases, EGTA was added to 5 mM and control/conditioned-media was at a final dilution of 1:5.

For harvesting, cells were washed twice with phosphate-buffered saline (PBS) and lysed in RIPA buffer (1% TRITON®-X100, PBS, 10 mM EDTA, 150 mM NaCl, 1% sodium-deoxycholate, 0.1% SDS, protease inhibitors). After determining protein concentrations (BCA; Pierce, Rockford, Ill.) for equal loading, SDS-PAGE and immunoblot analyses were performed with mouse anti-tubulin (Sigma, St. Louis, Mo.), rabbit anti-Lef1 (U072), and mouse anti-β-catenin (Sigma, St. Louis, Mo.). HRP-conjugated secondary-antibodies were followed by enhanced chemiluminescence (Amersham, Piscataway, N.J.).

BMP/Noggin Expression Analyses. RNAs were purified by Trizol-extraction of keratinocytes (INVITROGEN™, Carlsbad, Calif.) and spin-column chromatography (QIAGEN®, Valencia, Calif.). Reverse transcription (RT) was performed with a Superscript kit (INVITROGEN™, Carlsbad, Calif.). BMP2, BMP4, noggin, and GAPDH mRNAs were detected by RT-PCR. For BMP immunodetection, 1 ml of spent-keratinocyte or control media were precipitated with two volumes of acetone, while keratinocytes were pelleted directly by centrifugation. Samples were subjected to 10% SDS-PAGE and immunoblotted with anti-BMPs (R&D Systems).

Transfections. FUGENE6™ (Roche, Indianapolis, Ind.) was used to transfect newborn skin keratinocytes with expression vectors. For β-galactosidase reporters, CMV-luciferase was used to control for transfection efficiency, and for luciferase reporters, CMV-β-galactosidase was used. After 24 hours, cells were treated with Wnt and/or noggin media as described. β-Galactosidase activity was measured with the Galacto-Lite-Assay Kit (Tropix Inc.) and luciferase activity by the Dual-Luciferase Kit (Promega, Madison, Wis.). For standardization, the activity in the transfected lysate of control cells was assigned an arbitrary value.

Histology and Electron Microscopy. Tissues for immunofluorescence and hematoxylin and eosin staining were embedded in OCT and then frozen immediately on dry ice.

For transmission electron microscopy, samples were fixed in 2% glutaraldehyde, 4% formaldehyde, and 2 mM CaCl₂ in 0.05 M sodium cacodylate buffer for more than one hour at room temperature and processed for Epon embedding as described (Segre, et al. (1999) supra). Samples were visualized with a Tecnai G2 transmission electron microscope.

Tissues for scanning electron microscopy were fixed in 2.5% glutaraldehyde in phosphate buffered saline (PBS) for more than one hour at room temperature, processed using standard techniques (Fujiwara, et al. (2002) Development 129:4685-96), and visualized using a JEOL JSM-35 scanning electron microscope.

In Situ Hybridization. Digoxygenin-labeled probes were synthesized with the DIG-RNA labeling kit and detected with Anti-Digoxygenin AP (both from Roche, Indianapolis, Ind.). Antisense E-cadherin cRNA was generated from T7 RNA polymerase transcription of a BglII-XhoI extracellular domain fragment that had been subcloned into pCRII (INVITROGEN™, Carlsbad, Calif.) and linearized with HindIII. Sense probe was generated by XhoI digestion and Sp6 RNA polymerase transcription. 3′-UTR P-cadherin cRNAs were generated by subcloning a 379 bp PCR product into pCRII. Probes were applied to 10-15 μm sections of frozen, OCT-embedded tissue and processed using standard methods (DasGupta and Fuchs (1999) supra).

Whole mount in situ hybridization was performed using well-known methods (Conlon and Rossant (1992) Development 116:357-68) with some modifications (Byrne, et al. (1994) Development 120:2369-83). In situ hybridizations on 10 μm (Shh) and 15 μm (GATA-3) frozen sections were performed using standard methods (Schaeren-Wiemers and Gerfin-Moser (1993) Histochemistry 100:431-40).

X-Gal Staining and Immunofluorescence. OCT sections of indicated thickness were briefly fixed (30 seconds) in 0.1% glutaraldehyde, washed three times in phosphate buffered saline (PBS), and incubated in X-gal staining solution for 1-2 hours at 37° C.

Standard procedures were used for immunofluorescence staining. In short, OCT sections of indicated thickness were fixed for 10 minutes in 4% paraformaldehyde in PBS and washed four times five minutes in PBS. When staining with mouse monoclonal antibodies, the reagents and protocols used were from the MOM basic kit (Vector Labs, Burlingame, Calif.). In other cases, the following block/diluent was used: 2.5% normal donkey serum, 2.5% normal goat serum, 1% bovine serum albumin, 2% gelatin, and 1% TRITON® X-100 in PBS.

The following primary antibodies were used at the indicated concentrations: AE13 (mouse, 1:50-1:100; Lynch et al. (1986) supra), AE15 (mouse, 1:10; O'Guin et al. (1992) supra), K5 (gp, 1:250), GATA-3 (mouse, 1:100) and BMPR1A (1:100; Santa Cruz Biologicals, Santa Cruz, Calif.), β-galactosidase (rabbit, 1:400; mouse 1:100; Harlan, Indianapolis, Ind.; SIGMA, St. Louis, Mo.), Lef1 (rabbit, 1:250), K6 (rabbit, 1:500), Ki67 (rabbit, 1:1000; NovoCastra Laboratories Ltd, Newcastle, UK), K1 (rabbit, 1:250), loricrin (rabbit, 1:300), Lef-1 (rabbit, 1:250), filaggrin (rabbit, 1:1000; Covance, Harrogate UK), involucrin (mouse, 1:200; BabCO, Richmond, Calif.), rat anti-E-cadherin, rat anti-P-cadherin (Zymed), FOG1 (goat, 1:50; Santa Cruz Biotechnology, Santa Cruz, Calif.), guinea pig anti-K5, rat anti-HA (Roche, Indianapolis, Ind.) and rabbit anti-laminin5 (8LN5). Secondary FITC or Texas Red conjugated donkey or goat antibodies (1:100) (Jackson Laboratories, Bar Harbor, Me.) were used for detection of primary antibodies.

For detection of nuclear β-catenin, whole embryos were fixed overnight in 4% PFA, dehydrated, embedded in paraffin, and rehydrated prior to probing with mouse anti-β-catenin antibody (Clone 15B8, Sigma Chemicals, St. Louis Mo.).

Expression of TOPGAL was determined by X-gal staining of frozen embryo sections (7.5 μm) embedded in OCT and fixed in 0.1% glutaraldehyde (DasGupta and Fuchs (1999) supra). Histological analysis of embryonic skin was performed by staining with hematoxylin and eosin (Richard Allan Scientific).

CHIP and PCR. Protein-DNA complexes were crosslinked in whole cells with 1% formaldehyde, followed by sonication to fragment genomic DNA to a mid-range of 600 bp. DNA from anti-Lef1 immunoprecipitation was subjected to PCR using Taq-polymerase (Promega, Madison, Wis.) and primers specific for a 123 bp sequence encompassing the Lef1 binding site (Lef1 site; 5′-CAAAGAAAATAAAAACATAAGAAAC-3′, SEQ ID NO:9; 5′-TCCTATTCCACGGTCGTTCG-3′, SEQ ID NO:10 (Huber, et al. (1996) supra)) and a site ˜2 kb upstream (5′ site; 5′-AGCACCTCTATAGATGAGGC-3′, SEQ ID NO:11; 5′-TACTAAGGCCAAAACAATCACTG-3′, SEQ ID NO:12). PCR was performed with 40 cycles of denaturation at 94° C. for 30 seconds, annealing at 55° C. for 45 seconds, 72° C. for 30 seconds. PCR products were separated on 1.5% agarose gels and visualized with ethidium bromide.

Barrier Function Assay. Embryos were submerged for more than 8 hours at 37° C. in a solution of 1.3 mM MgCl₂, 100 mM NaPO₄, 3 mM K₃Fe(CN)₆, 3 mM K₄Fe(CN)₆, 0.01% sodium deoxycholate, 0.2% NP40, and 1 mg/mL X-gal which was adjusted to a pH of 4.5 with HCl (Hardman, et al. (1998) Development 125:1541-52). At this pH in the absence of the epidermal barrier, the solution penetrates epidermis, and an endogenous β-galactosidase-like activity catalyzes production of a blue precipitate.

Skin Grafting. Neonatal Bmpr1a conditionally null animals were identified by their lack of whisker follicles, and were sacrificed along with their wild-type littermates. Full thickness skins were removed from the torsos of wild-type and null embryos, spread on a sterile plastic dish, and stored briefly at 4° C. During this time, each skin graft recipient site was prepared by removal of a patch of full thickness skin on an anesthetized female nu/nu CD-1 mouse. Donor skin was then placed on the graft bed and secured by sterile gauze and cloth bandages. Each showed a consistent phenotype dependent on the presence or absence of BMPR1A in the donor skin.

Pregnant GATA-3nlslacZ/+dams were sacrificed at E17.5, and embryos were removed. Full thickness skins were removed from torsos of wild-type and GATA-3 null embryos, spread on a sterile plastic dish, and stored briefly at 4° C. During this time, each skin graft recipient site was prepared by removal of a patch of full thickness skin on an anesthetized female nu/nu CD-1 mouse. Embryonic donor skin was then placed on the graft bed and secured by sterile gauze and cloth bandages. A total of twelve grafts were placed (six wild-type and six GATA-3 null), and each showed a consistent phenotype dependent on the presence or absence of GATA-3 in the donor skin.

### Example 2

### E-cadherin Regulation is Dependent on Wnt and BMP Inhibitor Signaling

It was found that Wnts, expressed in ectodermal buds (St-Jacques, et al. (1998) Curr. Biol. 8:1058-68; Reddy, et al. (2001) Mech. Dev. 107:69-82), stabilize β-catenin at these sites. Canonical skin Wnt3a was tested for its ability to generate nuclear β-catenin in mouse keratinocytes. Keratinocytes exposed to Wnt3a-conditioned media displayed an ˜7× increase in β-catenin as judged by immunoblot and densitometry analysis. This increase was paralleled by accumulation of β-catenin in ˜85% of the nuclei of treated cells.

Wnt-treated cultures did not express appreciable Lef1, indicating the need for additional signaling molecules to induce a DNA binding protein for β-catenin transcriptional activity. As bud formation in vivo requires a mesenchymal cue (Hardy (1992) supra), candidates expressed by developing dermal condensates were analyzed. Epithelial cells and mesenchymal cells within follicle buds express BMPs 2 and 4 (Kratochwil, et al. (1996) Genes Dev. 10:1382-94; Kulessa, et al. (2000) EMBO J. 19:6664-74), while only mesenchymal cells express their inhibitor, noggin (Botchkarev, et al. (1999) Nat. Cell Biol. 1:158-64). Reverse transcriptase PCR and western analyses revealed that in vitro, keratinocytes express BMPs, but not noggin. When exposed to noggin-conditioned media, Lef1 was induced, and localized to the nucleus.

Together, Wnt and noggin promoted transactivation of the TOPFLASH reporter gene, which regulated luciferase expression through Lef/Tcf binding sites in the TOP promoter (Brantjes, et al. (2002) supra). When the Tcf/Lef1 binding sites were mutated (FOPFLASH), promoter activity was abolished, as it was when noggin was absent and BMPs were present. Additionally, although some stabilization of cytoplasmic β-catenin occurred naturally in keratinocyte cultures and in noggin treated cells, transactivation was clearly enhanced by Wnt3a. Thus, in vitro, noggin and Wnt3a appeared to act in concert to generate transcriptionally competent Lef1 complexes.

Noggin and Wnts also appeared to be essential for this process in vivo. In E16.5 skin, nuclear Lef1 and β-catenin could be seen in most follicles except for the mature guard follicles developing at E13.5 independently of Lef1. Lef1 positive hair buds also expressed β-galactosidase under the control of the TOP promoter (DasGupta and Fuchs (1999) supra). In contrast, noggin(−/−) mice lacked two thirds of E16.5 buds (Botchkarev, et al. (1999) supra), and those that formed exhibited cytoplasmic β-catenin and little or no Lef1 or TOPGAL activity. When noggin (−/−)/TOPGAL mice were mated on a background of K14-Lef1 (Zhou, et al. (1995) supra) to force Lef1 expression, nuclear β-catenin and TOPGAL expression were restored in ˜80% of the E16.5 buds. However, follicles were not restored to wild-type levels, indicating that some of the altered genes and morphology associated with noggin(−/−) follicles are not under control of Lef1/β-catenin, but possibly by other genes affected by the absence of noggin (Botchkarev, et al. (1999) supra).

When cells of embryonic epidermis (epi) reorient to form an epithelial bud, they switch from E-cadherin to P-cadherin based AJs. This timing coincided with nuclear Lef1 expression and TOPGAL activation. At the mesenchymal-epithelial interface, the cadherin switch persisted throughout follicle development, and was still in Lef1-positive, stem cell progeny of adult follicles. As in buds, adult hair precursor cells were not only E-cadherin dim and β-cadherin bright, but also active for nuclear Lef1, β-catenin and TOPGAL (DasGupta and Fuchs (1999) supra; Merrill, et al. (2001) Genes Dev. 15:1688-705). It has been noted that in developing mouse brain, E-cadherin was absent wherever Wnt-1 was expressed (Shimamura, et al. (1994) Development 120:2225-34). A correlation between reduced E-cadherin and elevated nuclear β-catenin has also been observed in vitro and in human cancers (Conacci-Sorrell, et al. (2002) J. Clin. Invest. 109:987-91).

In situ hybridization revealed that the cadherin switch was regulated, in part, at the mRNA level. Both cadherin promoters harbor multiple sequence motifs corresponding to the optimal Lef1/Tcf binding site, and the E-cadherin promoter has been shown to bind recombinant Lef1 protein (Huber, et al. (1996) Mech. Dev. 59:3-10). Although P-cadherin appeared unaffected by a noggin or Lef1 null background, E-cadherin mRNA and protein failed to be downregulated, a feature which was restored when mice were bred on the K14-Lef1 background. These data provided the E-cadherin gene as the first example of a candidate for negative repression by noggin-induced Lef1.

To further assess the extent to which E-cadherin downregulation correlated specificallywith follicle downgrowth, E16.5 skin null for sonic hedgehog (Shh), which when absent impairs follicle development soon after Lef1/β-catenin activation (St-Jacques, et al. (1998) supra), were analyzed. Arrested Shh (−/−) hair buds were positive for P-cadherin, and suppressed E-cadherin at both protein and mRNA levels. Taken together, these data place noggin involvement earlier than Shh, and specifically required for E-cadherin downregulation at the onset of hair morphogenesis.

It was determined if the negative effects of Lef1 on E-cadherin mRNA expression could be independent of β-catenin. E-cadherin expression in skin from a mouse expressing a constitutively stable β-catenin was examined. In interfollicular epidermis, the transgene (K14ΔNβ-catenin) elicits de novo follicle-like downgrowths (Gat, et al. (1998) supra), which display evidence of Wnt-responsive gene transactivation on a TOPGAL background (DasGupta and Fuchs (1999) supra). E-cadherin downregulation was consistently observed only at Lef1 positive sites of K14ΔNβ-catenin induced epithelial invaginations. A complementary study on Wnt-1 null mice reported a broadening of the E-cadherin pattern in developing brain tissue. These data indicate the dual and atypical importance of both stabilized β-catenin and Lef1 in repressing E-cadherin expression.

To analyze the observed changes in E-cadherin mRNA expression at the transcriptional level, a 6.5-kb fragment of the 5′ murine E-cadherin gene sequence was cloned and engineered to contain a mutation in the promoter that had previously been shown by gel shift assays to bind recombinant Lef1 (Huber, et al. (1996) supra). When tested in primary mouse keratinocytes, both wild-type and mutant promoters yielded comparable β-galactosidase reporter gene expression, indicating that the Lef1/Tcf site is not required for basal promoter activity.

Transfection of either stable β-catenin or Lef1 on their own did not appreciably affect the activity of the E-cadherin promoter. In contrast, Lef1 and stabilized β-catenin in combination markedly suppressed E-cadherin promoter activity in a manner dependent upon the Lef1/Tcf binding site. Similar repression was observed when noggin and Wnt3a together were added to the keratinocyte cultures.

To determine whether Wnt-activated Lef1 complexes bind to the endogenous E-cadherin promoter, chromatin immunoprecipitation analyses (ChIP) on Lef1-expressing keratinocytes were conducted. Only when these cells were exposed to Wnt3a were 120 bp chromatin fragments of the E-cadherin promoter containing the Lef1 binding site specifically precipitated. In contrast to the repressive effects on the E-cadherin promoter, Wnt and noggin together activated the murine HK1-hair keratin promoter, previously identified as a bona fide Lef1/β-catenin responsive target in keratinocytes (Merrill, et al. (2001) supra).

These findings indicate that Lef1's ability to function as repressor and activator in the same Wnt-treated cells depends upon the context of responsive promoter elements. Located 3′ from the Lef1 motif in the E-cadherin promoter is an E-box sequence, which in various cell lines serves as the binding and regulatory site for the Snail family of transcriptional repressors (Cano, et al. (2000) Nat. Cell Biol. 2:76-83; Battle, et al. (2000) Nat. Cell Biol. 2:84-9). Mutation of the Lef1 binding site did not interfere with Snail's ability to repress the E-cadherin promoter. Moreover, these two sites together functioned additively to repress wild-type E-cadherin promoter activity by up to 70% its normal activity in keratinocytes.

Despite the presence of functional in vitro binding sites for Snail and Lef1, the E-cadherin promoter may still be regulated in vivo by indirect pathways involving these factors. Irrespective of mechanism, the independent repressor action of Snail was of interest given that the large guard hair follicles that develop on both the noggin and Lef1 null genetic backgrounds still exhibited E-cadherin downregulation. These data reveal E-cadherin downregulation as a common thread among the waves of follicle morphogenesis and underscore the existence of multiple mechanisms to govern this downregulation.

To determine the functional importance of E-cadherin downregulation in hair follicle morphogenesis, transgenic mice were engineered to express elevated levels of an epitope-tagged E-cadherin. Previous studies have shown that the addition of a C-terminal tag does not interfere with E-cadherin's ability to form intercellular junctions (Adams, et al. (1998) J. Cell Biol. 142:1105-1119). Several of the newborn animals harboring the K14-Ecadherin-HA transgene were sickly, and were euthanized shortly after birth. Immunofluorescence analyses of frozen newborn skin sections revealed a mosaic pattern of anti-HA negative and positive domains. Signs of the characteristic E-cadherin downregulation were seen in the non-transgenic (Tg−) epithelium, but were not detected in transgene positive (Tg+) areas. Hematoxylin and eosin staining confirmed that the most striking morphological difference between Tg− and Tg+ regions was the paucity of hair follicles in Tg+ skin.

Skin, conditionally null, for α-catenin (by K14-Cre) also lacked proper adherens junctions (Vasioukhin, et al. (2001) Cell 104:605-17). At E16.5, developing placodes were visible, but they were severely arrested by birth. Correspondingly, sebaceous glands failed to develop. These findings demonstrate the deleterious consequences of too few as well as too many adherens junction proteins in hair follicle morphogenesis, and demonstrate that dynamic changes in AJ gene expression is a key step in morphogenesis. Either complete loss or overexpression of E-cadherin can also impair formation of intestinal epithelia and mammary glands (Hermiston, et al. (1996) Genes Dev. 10:985-96; Boussadia, et al. (2002) Mech. Dev. 115:53-62). In contrast, a reduction in adherens junctions is characteristic of epithelial cancers, whose cellular masses bear some resemblance to aberrant bud formation (Conacci-Sorrell, et al. (2002) supra).

### Example 3

### GATA-3 and Stem Cell Lineage Determination

Gene expression profiles of murine dorsal skin at three critical times during embryogenesis (E13, E15, E18.5) were analyzed to identify genes involved in hair follicle morphogenesis. The transcriptional regulator GATA-3 was found to be induced at E15 at the early stages of hair follicle placode formation and sustained in expression as the IRS begins to develop at E18.

GATA-3 is a member of the GATA family of zinc finger transcription factors, which play key roles in controlling cell fate decisions, in particular in different hematopoietic lineages (Cantor and Orkin (2002) Oncogene 21:3368-76; Kuo and Leiden (1999) Annu. Rev. Immunol. 17:149-87). Early in lymphoid development, GATA-3 is essential for the T lymphoid cell lineage, while later, it is critical for differentiation of naive CD4+ T cells into Th2 as opposed to Th1 effector cells (Ting, et al. (1996) Nature 384:474-8; Hendriks, et al. (1999) Eur. J. Immunol. 29:1912-8, Zheng and Flavell (1997) Cell 89:587-96; Zhang, et al. (1997) J. Biol. Chem. 272:21597-603). The induction of GATA-3 was of interest as Lef-1, which is involved in hair shaft lineage determination, was named as lymphoid enhancer factor 1 (Lef-1) on the basis of its initially described role in T cell development, where it regulates T-cell receptor genes (Carlsson, et al. (1993) Genes Dev. 7:2418-30; Giese and Groschedl (1993) EMBO J. 12:4667-76; van Genderen, et al. (1994) Genes Dev. 8:2691-703). While ablation of GATA-3 results in severe defects in thymic and T-cell development, embryonic lethality arises due to noradrenaline deficiency and cardiac defects, with additional abnormalities in cephalic neural crest and renal development (Pandolfi, et al. (1995) Nat. Genet. 11:40-4; Lim, et al. (2000) Nat. Genet. 25:209-12). Skin has not been examined in GATA-3 null embryos which die by E11.5 without pharmacological rescue, prior to hair follicle morphogenesis.

As GATA-3 was induced in E13-18.5 back skin and skin has been included in a general list of GATA-3 positive organs (Oosterwegel, et al. (1992) Dev. Immunol. 3:1-11; Lakshmanan, et al. (1999) Mol. Cell Biol. 19:1558-68), the temporal and spatial expression pattern of GATA-3 was analyzed. As judged by whole mount in situ hybridizations, GATA-3 cRNA hybridization was detected in the early developing vibrissae follicles by E14.5. In body skin, hybridization was first detected at E15.5 and by E17, was evident both in the developing epidermis and hair follicles. These expression patterns extended the microarray analyses and revealed an induction of GATA-3 mRNAs in what appeared to be terminally differentiating cells of epidermis and the cone of presumptive IRS precursor cells (Pre-IRS) within developing follicles. The IRS develops somewhat earlier than the hair shaft, and precortical cells are not yet present at this stage.

To further analyze the temporal and spatial transcriptional changes in GATA-3 expression, an engineered GATA-3nlslacz mouse line was used, in which a gene encoding a nuclear-targeted version of β-galactosidase had been knocked in to the GATA-3 locus (Hendriks, et al. (1999) supra, van Doorninck, et al. (1999) J. Neurosci. 19:RC12). In mice heterozygous for this allele, β-galactosidase expression was absent in the E13 single-layered keratin K5-expressing embryonic epidermis but became evident in the newly formed suprabasal layer of E15 epidermis. Although β-galactosidase and GATA-3nlslacZ expression were either absent or weak within the E15 embryonic basal layer, it was restored in this layer of newborn epidermis and remained on thereafter. Overall, expression patterns of GATA-3 mRNAs and GATA-3 promoter-driven nlslacz activity were similar, if not identical.

By postnatal day 16 (P16) when back skin follicles are mature and nearing completion of the first growth (anagen) phase of the hair cycle (Hardy (1992) Trends Genet. 8:55-61), GATA-3 was clearly expressed in follicles as well as epidermis. Shortly after the synchronous initiation of the first postnatal hair cycle, X-gal staining was seen in a cone, distinctive of the organization of Pre-IRS, composed of IRS progenitor cells.

The precise location of GATA-3 expression within the hair follicle was determined by comparing GATA-3 promoter and protein expression to the expression of well established biochemical markers for follicles. As judged by both GATA-3 promoter-driven β-galactosidase expression and GATA-3 protein expression, GATA-3 expression was restricted to two cell layers of the follicle. These layers were located directly external to the hair shaft cuticle and cortex/precortex, marked by an AE13 monoclonal antibody specific for hair-specific keratins (Lynch, et al. (1986) J. Cell Biol. 103:2593-606). At the base of the follicle, the two β-galactosidase-positive layers were directly internal to a single layer of IRS cells labeling with the monoclonal antibody AE15 specific for trichohyalin, a marker of IRS and postnatal, differentiating medulla cells (O'Guin, et al. (1992) J. Invest. Dermatol. 98:24-32). Further up, the two β-galactosidase-positive cell layers co-labeled with AE15 antibodies, which now detected all three IRS layers.

Further, a single layer of unlabeled nuclei (Henle's layer) was present between the K6-positive companion cell layer and the two GATA-3-positive Huxley's and cuticle layers of IRS. Although some K6 genes are also expressed in IRS (Winter, et al. (1998) J. Invest. Dermatol. 111:955-62; Langbein, et al. (2003) J. Invest. Dermatol. 120:512-22), the K6 antibody used herein did not recognize these isoforms. In addition, two cell layers, namely hair shaft cuticle and cortex, separated AE15 and K6-positive medulla from GATA-3 positive IRS cuticle.

An analysis of GATA-3 null epidermis in E18.5 embryos, the latest stage to which these animals survive, was conducted. At this stage, back skin epidermis is mature, and most waves of follicle morphogenesis have initiated. However, the most mature follicles in the E18.5 embryo are vibrissae, so whisker pads were examined.

Whiskers of control littermates were visible on E18.5 embryos, and hematoxylin and eosin staining of wild-type vibrissae sections revealed morphological signs reflective of mature follicles. In E18.5 wild-type vibrissae, the precortex was labeled with AE13 antibodies, indicative of hair keratin expression. Analogous to postnatal follicles, several cell layers separated AE13-positive hair shaft cells from the K6-positive companion cell layer. Serial sectioning revealed that cells between the hair shaft and companion cell layer are IRS cell layers, which stained positively for the trichohyalin antibody AE15.

In E18.5 GATA-3 null vibrissae, AE13 (hair keratin)-positive cells appeared to directly abut the K6-positive cells of the companion cell layer. By staining alternating serial sections with AE13 and AE15 antibodies, it was clear that AE15-positive cells, characteristic of IRS, were either absent or severely reduced in GATA-3 null E18.5 vibrissae. In addition, the AE13-positive hair shaft and associated precursor layers appeared to be expanded in GATA-3 null follicles relative to wild-type follicles. These abnormalities were seen in all vibrissae, irrespective of their location within the GATA-3 null whisker pad.

Morphological aberrations in GATA-3 null vibrissae follicles included atypically bent shapes, strange nodular thickenings, and irregularities in the thickness of the ORS and bulb. These perturbations were manifested at the skin surface by a delayed eruption of GATA-3 null relative to wild-type vibrissae shafts.

Elsewhere within E18.5 skin, few differences were noted. As a measure of epidermal function, E18.5 knockout embryos were able to exclude blue dye, indicating that the epidermal barrier was intact. Analyses of E17.5 embryos revealed a slight delay in barrier function acquisition at this earlier age, as judged by the complete penetration of blue dye through GATA-3 null skin relative to only partial penetration through wild-type E17.5 skin. Overall, however, the morphology and biochemistry of GATA-3 null epidermis throughout E18.5 embryos was largely similar to wild-type epidermis. Thus, basal, spinous, granular, and cornified layers were all present and of comparable size and thickness. Wild-type and knockout epidermis displayed normal staining for the basal layer keratin 5, the suprabasal layer keratin 1 and the late-stage differentiation markers involucrin, loricrin, and filaggrin.

Morphological and biochemical signs of IRS and hair shaft differentiation were largely absent in back skin prior to birth. The lack of overt abnormalities in the back skin of even the oldest living GATA-3 knockout embryos explains why skin defects had not been noted previously in these animals (Lim, et al. (2000) supra).

A priori, the selective abnormalities in E18.5 GATA-3 null vibrissae could be attributed either to a special role for GATA-3 in vibrissae or to their early development. In addition, given the well-established role of GATA-3 in hematopoiesis and CNS/PNS development, it was possible that the hair follicle defects were attributable to indirect rather than autonomous effects. To distinguish between these possibilities, full thickness E17.5 skin from GATA-3 null and wild-type littermate embryos were grafted onto backs of recipient nu/nu mice. Mice with the nu/nu mutation are immunocompromised as they lack T cells and cannot reject the graft. They also lack nearly all external hair shaft formation, but are wild-type for GATA-3 (Flanagan (1966) Genet. Res. 8:295-309; Nehls, et al. (1994) Nature 372:103-7; Segre, et al. (1995) Genomics 28:549-59).

When bandages were removed from recipient mice at 7 days after engraftment, hair shafts were already evident on wild-type grafts but not on GATA-3 null grafts. Grafts were monitored daily, and in six of six grafts, knockout skin consistently failed to develop the thick hair coat displayed by wild-type grafts. Instead, knockout grafts developed short “stubble” that failed to progress into a normal hair coat.

In wild-type grafts, all three major hair types, including awl, guard, and zig-zag hairs were readily identified upon microscopic examination of plucked hairs. In contrast, hairs from GATA-3 null grafts were shorter and wider and did not fall into any standard classification. As judged by scanning electron microscopy, the fine structure of these hairs was highly irregular. Wild-type follicles displayed a “shingle-like” pattern, characteristic of flat cuticle cells that constitute the outer layer of normal hair shafts. While flat cuticle cells were present in knockout shafts, their organization was highly abnormal. Additionally, knockout hair shafts were short, and their diameter was large and irregular compared to their wild-type counterparts. The nodules, bulges and bends in GATA-3 null hairs paralleled the follicle defects seen below the skin surface in embryonic GATA-3 null vibrissae follicles.

The skin grafts enabled the examination of changes in expression patterns of hair follicle proteins in mature pelage follicles, replete with hair shafts. Immunofluorescence analyses revealed severe abnormalities in the expression patterns of hair follicle proteins within GATA-3 null skin grafts. As judged by AE13 staining to detect the cortical keratins of hair shafts, GATA-3 null follicles exhibited a markedly expanded precortex and cortex as compared with wild-type grafts. These differences were further documented by a large increase in Lef-1 positive precortical cell nuclei in knockout versus wild-type follicles. In addition, there was an expansion of differentiated medulla cells expressing trichohyalin, which was flanked by the AE13-positive cortex. Ultrastructural analyses confirmed the identity of these cells as medulla rather than a misplaced core of IRS cells.

Increases in cortical and medulla cells were paralleled by a paucity of differentiated IRS cells labeled with AE15 antibodies against trichohyalin. Thus, in contrast to the two robust stripes of wild-type IRS stained with AE15 antibodies, only wisps of AE15-positive knockout IRS cells were detected. Additionally, these remnants of what appeared biochemically to be IRS were found well above the bulb of the follicle and above the initiation of medulla differentiation. In marked contrast, IRS differentiation in wild-type grafts initiated in a horizontal plane near the base of the bulb, comparable to that of the Lef-1-positive cortical precursors and well below the differentiating medulla.

Despite the paucity of trichohyalin-positive IRS cells of GATA-3 null grafted skin, the K6-positive companion layer appeared to be largely normal in size and location. However, whereas the companion layer in wild-type follicles flanked the AE13-negative/AE15-positive IRS cells, it flanked the AE13-positive/AE15-negative cortical cells in knockout follicles. These results indicated that differentiating IRS cells were largely absent in GATA-3 null follicles.

The presence of a large group of Lef-1-negative epithelial cells surrounding the Lef-1-positive cortical precursor cells near the base of the hair bulb was indicative of these Lef1-negative cells being IRS precursors. This was examined by using the functional LacZ coding sequence inserted downstream from the GATA-3 promoter of the knockout animals. Previously, it had shown that GATA-3nlslacZ+/−follicles displayed a pattern of β-galactosidase activity that paralleled the two rows of anti-GATA-3 immunoreactivity demarcating the Huxley's and cuticle layers of the IRS. The expression pattern of GATA-3 promoter activity was analyzed under conditions where GATA-3 protein was missing.

Unexpectedly, GATA-3 null follicles displayed a gross expansion of GATA-3 promoter-active (β-gal positive) cells. Thus, rather than two neat rows as in wild-type follicles, extensive GATA-3 promoter activity was detected within cells of the follicle bulbs of GATA-3 protein-deficient follicles. Most of these cells were negative for AE13 and AE15-staining. Thus, neither their position, nor their expression pattern was consistent with a precortical identity.

Markers for characterizing the GATA-3 promoter active bulb cells were examined. For example, the expression of FOG1 (Friend of GATA-1) was analyzed. FOG often functions with GATA factors, including GATA-3, to suppress GATA's action as an activator (Tsang, et al. (1997) Cell 90:109-19; Zhou, et al. (2001) J. Exp. Med. 194:1461-71). In wild-type follicles, an anti-FOG1 antibody labeled the same two IRS layers that labeled with GATA-3 antibodies. Antibodies against GATA-3 and FOG1 both localized to the nuclei of these wild-type IRS precursor cells. In GATA-3 null follicles, anti-FOG1 costained with anti-β-galactosidase antibodies in the expanded zone of GATA-3-promoter active cells. In the absence of GATA-3, however, anti-FOG labeling shifted from nucleus to cytoplasm, indicating that GATA-3 and FOG-1 may function together at some point in IRS specification.

The increased population of Lef1-positive pre-cortical cells in GATA-3 null follicles indicated an interdependency of the development of IRS and cortical progenitors. Given the marked aberrations in hair shaft, however, a determination of whether the expanded compartments of IRS and cortical precursors were truly normal. Closer inspection of GATA-3 promoter active cells revealed a number of cells whose nuclei were positive for both β-galactosidase and cortical keratins. Conversely, some of the Lef1-positive cortical precursor cells were also positive for β-galactosidase. Such mixing of biochemical markers was not seen in wild-type follicles, where Lef1-positive precortical cells were distinct in their expression pattern from the compartment of GATA-3/FOG1/GATA-3-promoter-active IRS precursor cells.

Given the expansion of precursor compartments for GATA-3 null IRS and cortex, the status of matrix cells, which are thought to give rise to these precursor populations, were examined. In both wild-type and knockout follicles, antibodies against the proliferating nuclear antigen, Ki67, revealed intense labeling of the matrix cells, which in anagen, are rapidly proliferating. Overall, the number of Ki67-positive cells was similar in wild-type and knockout follicles.

The status of Sonic hedgehog (Shh), a regulator of follicle morphogenesis that is normally expressed in a small zone of matrix cells (Gambardella, et al. (2000) Mech. Dev. 96:215-8) was analyzed. In mutant mice deficient in the CCATT box displacement factor, CDP, Shh mRNAs are absent from this zone and instead activated in the region corresponding to the IRS (Ellis, et al. (2001) Genes Dev. 15:2307-19). Since both CDP and GATA-3 mutations lead to IRS abnormalities, it was determined whether such misregulation might occur in the absence of GATA-3 as well. The expression of Shh mRNAs appeared to be unaffected in the GATA-3 mutant follicles.

Ultrastructural analysis of wild-type and GATA-3 null graft skin by transmission electron microscopy further defined abnormalities. An analyses of wild-type and GATA-3 null embryonic skins demonstrated that differences in epidermises of grafted wild-type and GATA-3 null skin were relatively slight, and expression patterns of cornified envelope proteins and keratins 1 and 5 were similar. Morphologically, GATA-3 null epidermis displayed all four stages of terminal differentiation, but the tissue was hyperthickened, consistent with the mild barrier function defect observed earlier. More robust signs of barrier defects have been observed in epidermis lacking the Kruppel-like transcription factor, KLF4 (Segre, et al. (1999) Nat. Genet. 22:356-60).

Abnormalities were striking between wild-type and knockout grafted skins. Follicles in wild-type grafted skin developed normally, extending deep into the dermis. In contrast, GATA-3 null follicles and their developing shafts were shorter and fatter, despite expanded compartments of IRS and cortical precursor cells in enlarged bulbs. This appeared to be reflective of defects in both the downward movement of developing follicles as well as upward movement of hair shafts.

At the ultrastructural level, wild-type grafted follicles displayed a normal IRS, with cuticle, Huxley, and Henle layer readily identified by electron dense trichohyalin granules. The Henle layer is the first IRS layer to accumulate trichohyalin granules, and it eventually keratinizes as it progresses up the follicle (Robins and Breathnach (1970) J. Anat. 107:131-46). At the core of this layer was a single row of highly organized medulla cells, which also displayed clusters of trichohyalin granules just below the nucleus.

In GATA-3 null follicles, a single layer of differentiated IRS-like cells were detected. Consistent with the anti-trichohyalin labeling, these cells were well above the bulb and contained only a few electron-dense trichohyalin granules. Thus, despite a significant pool of IRS precursor cells, morphological as well as biochemical signs of Huxley's and IRS cuticle cells were lacking.

Internal to the Henle's layer, only occasional trichohyalin-negative, immature cells were seen in the GATA-3 null follicles. Instead, most of the central portion of GATA-3 null follicles was packed with keratinized material characteristic of the hair shaft. Below the remnants of the Henle's layer were precortical-like cells, displaying bundles of keratin filaments.

In the shaft area of most GATA-3 null follicles, ORS/companion layer cells with wisps of keratin filaments were abutted against keratin filament-rich precortical-like cells, which in turn were adjacent to cortex and medulla. Additionally, medulla cells were markedly disorganized and often embedded in a mass of keratinized cortical cells. Some medulla cells displayed trichohyalin granules. However, the organization of granules as well as the organization of cells was aberrant. Overall, this analysis confirmed the medulla identity of the thickened core of AE15-positive cells in GATA-3 null follicles and revealed a level of disorganization of the hair shaft that was even more prominent than that envisioned from the immunohistochemical studies.

The structural consequences of IRS loss were severe. Without the Huxley's and cuticle layer, the Henle's layer did not develop properly, and without the IRS, gross abnormalities arose in composition and organization of cells within the hair shaft. Thus, not only were the cortex and medulla dramatically thickened, but in addition, cells no longer spatially aligned. The shaft developed as a hyperthickened mass of irregular medulla, cortical and cuticle cells, rather than a normal shaft. The GATA-3 knockouts clearly demonstrated that these two thin layers of IRS cells are critical for the proper differentiation and/or organization of the hair shaft.

In contrast, although GATA-3's absence resulted in major abnormalities in hair structure, involving both IRS and shaft, it was only expressed in two of the IRS layers. Therefore, severe hair shaft defects that occurred in the GATA-3 deficient mouse originated specifically from transcriptional aberrations within the IRS.

The highly restricted pattern of GATA-3 expression was unique, but it was most similar to that of the transcriptional repressor CDP, which represses genes regulated by CCAAT sequence motifs (Ellis, et al. (2001) supra). The Cutll gene encoding CDP seemed to be expressed earlier and more broadly in the IRS pathway, where it was found in the outermost layer of the lower ORS, in IRS progenitor cells of the matrix, and in all three IRS layers. A few mice harboring mutations in Cutl1 survived their lung defects long enough to exhibit a sparse and abnormal hair coat. The morphology of Cutl1 null hair follicles was unusual, as just above a relatively normally structured bulb, the IRS formed, but then both IRS and shaft eventually degenerated to form a large follicular cyst, soon appearing more similar to that of nude or even hairless mutant mice (Ellis, et al. (2001) supra).

While both Cutl1 and GATA-3 null follicles exhibited a failure to develop a proper IRS, GATA-3's absence affected cell fate specification within the hair follicle. A lack of GATA-3 resulted in an accumulation of IRS precursor cells that appeared to be dysfunctional, unable to differentiate into trichohyalin and keratin producing IRS cells and this led to an expansion of seemingly functional Lef-1-expressing precortical cells, which differentiated into hair keratin-producing cortical cells. Some of these Lef1-positive cells exhibited mixing of IRS and cortical gene expression patterns. Since such cells were not detected in wild-type, GATA-3-containing follicles, these findings indicate that the loss of GATA-3 resulted not only in expansion of selective cell populations within the follicle, but also in some misexpression of genes in these compartments. In contrast, Cut1 null follicles exhibited substantial misexpression of both trichohyalin and the companion layer K6 in the cortex, features which were not observed in GATA-3 null follicles, as well as strong expression of the Cutl1 promoter throughout the cortex. Furthermore, in Cut1 null vibrissae, Shh was aberrantly expressed in the IRS (Ellis, et al. (2001) supra), whereas in GATA-3 mutant mice, Shh expression appeared unaffected. It may still be possible that CDP and GATA-3 function in the same pathway to regulate IRS specification, but the target genes they affect must be at least partially non-overlapping.

In T-cell development, both GATA-3 and Lef-1/Tcf1 are necessary early to direct lymphoid precursors to become T-cells, and later in the lineage, GATA-3 acts independently of Lef1 in Th cell commitment (Hattori, et al. (1996) J. Exp. Med. 184:1137-47; Ting, et al. (1996) supra; Verbeek, et al. (1995) Nature 374:70-4; Schilham, et al. (1998) J. Immunol. 161:3984-91; van Genderen, et al. (1994) supra; Okamura, et al. (1998) Immunity 8:11-20; Hendriks, et al. (1999) supra). In naive Th cells, FOG1 and GATA-3 act to inhibit interleukin gene expression, and upon Th commitment, FOG1 is downregulated (Kurata, et al. (2002) J. Immunol. 168:4538-45). GATA-3, which autoactivates its own expression, is required for Th2 cell differentiation (Ouyang, et al. (2000) Immunity 12:27-37; Zhou, et al. (2000) supra), while suppression of GATA-3 promotes Th1 cell determination (Patient and McGhee (2002) Curr. Opin. Genet. Dev. 12:416-22).

Despite some differences, their exits transcriptional parallels between T-cell development and hair follicle differentiation. In the postnatal hair follicle, Lef-1/β-catenin and GATA-3 seem to act in parallel but distinct pathways governing cortical and IRS differentiation, respectively. Not to be bound to any particular mechanism, the expansion of the cortical lineage in the GATA-3 deficient state may be analogous to an increase in Th1 rather than Th2 cell differentiation, and the upregulation of GATA-3 promoter activity in GATA-3 deficient IRS precursor cells may be a sign that FOG1 and GATA-3 normally repress this promoter in wild-type IRS precursors. When taken together with the paucity of differentiated IRS cells, the expanded population of GATA-3 promoter active/FOG1-positive cells in GATA-3 deficient follicles may represent a population of stalled IRS precursors. The enhanced GATA-3 immunoreactivity as IRS precursor cells differentiate into IRS cells in wild-type skin is consistent with a transactivating role for GATA-3 later in IRS differentiation, perhaps analogous to Th2 cell differentiation. As structural genes for IRS differentiation are not expressed in the GATA-3 deficient state, despite the presence of progenitor cells, indicates a positive role for GATA-3 in their expression.

### Example 4

### BMPR1a and the Differentiation of Inner Root Sheath and Hair Shaft

Conditional mutations were introduced into the Bmpr1a locus by mating Bmpr1a flox/flox (fl/fl) mice (Mishina et al. (2002) Genesis 32:69-72) with transgenic mice that express Cre under the control of a K14 promoter (Vasioukhin et al. (1994) Proc. Natl. Acad. Sci. USA 96:8551-8556). Offspring from matings of Bmpr1a(fl/+), K14-Cre mice yielded litters of the expected numbers, genotype and Mendelian ratios. However, mice harboring two floxed alleles of Bmpr1a and K14-Cre were smaller in size and displayed markedly aberrant whiskers. Although the number of whiskers in knockout mice was nearly always less than their wild-type counterparts, this varied, and some knockout animals displayed no whiskers at all. As a measure of epidermal function, E18.5 knockout embryos and their wild-type counterparts were able to exclude blue dye, indicating that the epidermal barrier was intact. Overall, the neonatal mice conditionally targeted for disruption of Bmpr1a gene function could readily be sorted on the basis of their phenotype which was confirmed by their genotype.

As judged by immunofluorescence, the targeted ablation of Bmpr1a was nearly, if not fully, complete by E15.5. In contrast to wild-type skin, knockout skin revealed few, if any, traces of residual BMPR1A protein in the developing epidermal basal layer. By E17.5, BMPR1A-positive hair follicles were visible in wild-type backskin, and by P1, wild-type follicles revealed BMPR1A-positive cells in most, if not all, differentiation stages. Similar to other studies, expression was highest in ORS, matrix and precortex (Botchkarev et al. (1999) supra). The absence of BMPR1A in embryonic skin verified the success of targeting and revealed no unusual stability in Bmpr1a mRNA or protein that might interfere with further analysis.

The knockout animals used for immunofluorescence studies exhibited no whiskers at all. In examining BMPR1A expression, a correlation between the extent of BMPR1A mosaicism and the number of whiskers present was observed. Further analysis revealed that the aberrant whiskers were genetically mosaic, while whiskers and hairs failed to form in the complete absence of BMPR1A. Accordingly, conditional knockouts where the majority of the follicles displayed no traces of anti-BMPR1A staining were used for subsequent analysis.

To assess whether follicular defects extended to backskin, and to probe more deeply into the nature of the defects of Bmpr1a null mutants, the histology of wild-type and neonatal Bmpr1a null follicles at postnatal day two (P2) of development were examined. Toluidine blue-stained, semithin sections revealed gross abnormalities in these follicles. By this age, some wild-type backskin hairs began to display a well-differentiated structure, replete with ORS, IRS and hair shaft. Most notably, P2 knockout follicles exhibited an ORS and bulbar-like structure, but lacked the typical IRS and hair shaft. Overall, the follicles appeared immature, resembling those of late-stage embryonic skin, rather than postnatal skin. However, as knockout follicles extended downward, they wove in and out of the plane of sectioning. In contrast, knockout epidermis exhibited seemingly normal terminal differentiation.

By P4, differences in follicles were even more dramatic. P4 knockout follicles still showed no signs of a dark blue-stained cortex, a cross-striated medulla, or an IRS, all well-developed in wild-type P4 counterparts. In addition, the distinction between the dermal papilla and the matrix was less obvious than in wild-type follicles. Overall, aberrations in knockout follicles did not seem to be due to a developmental lag, but rather a molecular defect in the differentiation process.

Ultrastructural analysis of wild-type and Bmpr1a null skin was used to define the extent of the abnormalities. Wild-type P2 follicles sometimes displayed an immature core, as the medulla was still forming in younger follicles of this age. However, most P2 follicles did exhibit a keratinized cortex, packed with dense keratin filaments, and a well-developed IRS, whose appearance precedes the hair shaft (Robins and Breathnach (1970) J. Anat. 107:131-46). At early stages of normal terminal differentiation, all IRS layers displayed trichohyalin granules, which are larger and more abundant than in medulla. Progressively upward, the Henle's layer of the IRS becomes highly keratinized, making it obvious as an electronic dense row of flat, vertical cells.

In P2 Bmpr1a null follicles the ORS appeared largely normal. Internally to the ORS was a group of cells which possessed morphology and orientation similar to that of the companion layer. The follicle was encased by a dermal sheath, but the morphological distinction between the surrounding mesenchymal cells and the knockout follicle cells was otherwise difficult to make. With the exception of occasional Henle layer cells, only a few P2 follicles (˜1:100) displayed morphological signs of IRS cells and the number of such follicles correlated with the rare BMPR1A mosaic follicles.

At the base (bulb) of both wild-type and knockout follicles, the relatively undifferentiated cells of the matrix were present. The matrix displayed numerous mitoses, consistent with its proliferative status. In addition, a basement membrane, composed of a basal lamina and lamina densa, separated the inner strand of dermal papilla cells, which seems to maintain matrix cells as undifferentiated hair progenitors (Fuchs et al. (2001) supra; Millar (2002) supra). Otherwise, the difficulties that were faced in distinguishing mesenchymal and epithelial cells at the light microscopy level persisted at the ultrastructural level. Since the keratin 14 promoter is only active in the skin epithelial cells and not in dermal papilla cells (Vasioukhin et al. (1999) supra), the dermal papilla defect seemed to be a secondary consequence of BMPR1A loss.

Differences noted in P2 follicles were exaggerated by P4. At this stage, the medulla was well-developed in most wild-type follicles, and its highly organized row of cells at the center of the follicle were packed with trichohyalin and melanin granules. In contrast, the P4 knockout follicle stem consisted predominantly of ORS and companion layer cells.

To further characterize abnormalities in Bmpr1a null follicles, immunofluorescence microscopy studies were conducted. In wild-type follicles, IRS and medulla label with an AE15 monoclonal antibody, specific for trichohyalin (O'Guin et al. (1992) J. Invest. Dermatol. 98:24-32). In contrast, AE13 marks the hair-specific keratins, the major structural proteins of the hair shaft (Lynch et al. (1986) J. Cell Biol. 103:2593-2606). In wild-type neonatal P1 skin, follicles are often positive for both AE15 and AE13. In P1 Bmpr1a null skin, however, no AE15 or AE13 staining was observed. These findings were consistent with the ultrastructural data provided herein.

Similar differences were seen in P8 follicles, a stage where wild-type follicles were nearing their height of growth and differentiation. By this age, all wild-type backskin follicles displayed AE15 and AE13 staining, whenever sectioning was in the plane of these structures. In contrast, >90% of P8 knockout follicles lacked AE15 and AE13 reactivity. The occasional knockout backskin follicle that exhibited histological and biochemical signs of IRS and hair shaft production correlated with the low levels of BMPR1A mosaicism observed. These data revealed major biochemical abnormalities in IRS and hair shaft differentiation.

Antibodies against keratin 5 are specific for the epidermal basal layer and ORS of the normal hair follicle (Byrne et al. (1994) Development 120:2369-2383). Additionally, certain keratin 6 proteins are specific for the companion layer, separating the outer and inner root sheaths (Winter et al. (1998) J. Invest. Dermatol. 111:955-962). As judged by immunofluorescence, the expression of K6 appeared to be somewhat increased and expanded in the knockout versus wild-type follicles. This was consistent with the ultrastructural data provided herein.

Aberrations in IRS and hair shaft differentiation were further examined by determining the expression of the transcription factors known to regulate these programs. The transcription factor GATA-3 plays an essential role in the differentiation of IRS progenitor cells (Kaufman et al. (2003) Genes Dev. 17(17):2108-22), while hair-specific keratin genes possess regulatory sequences for the Lef1 transcription factor (Zhou et al. (1995) supra).

GATA-3 was markedly diminished in knockout follicles relative to their wild-type counterparts. In contrast, although knockout follicles exhibited a loss of AE13 immunoreactivity, they retained Lef1 expression. This further indicates that Lef1 expression in skin keratinocytes may depend upon inhibition of BMP signaling (Botchkarev et al. (1999) supra).

Although K14-Cre is not active in mesenchymal cells, it was noted that the dermal papilla in knockout follicles was not as condensed in wild-type, and hence expression of the major transcription factors known to be expressed in dermal papilla was examined. Both Lef1 and the mesenchymal transcriptional regulator Runx3 were expressed in both wild-type and knockout dermal papilla. Wild-type and knockout bulbs also exhibited a line of labeling with antibodies against β4 integrin, a component of hemidesmosomes which help to adhere matrix cells to the basement membrane at the dermal papilla boundary.

To examine proliferation status of the knockout follicle cells, an antibody against Ki67, a proliferating nuclear antigen that is active in cycling cells was employed. This antibody strongly stained the proliferating matrix cells of wild-type P8 follicles. The antibody also labeled the matrix of the knockout follicles, although the pattern of labeling was somewhat different from that seen in wild-type matrix. Additionally, as judged by in situ hybridization, expression of Sonic hedgehog (Shh) was still seen in knockout follicle bulbs. However, rather than the highly restricted, asymmetric pattern typical of Shh in wild-type bulbs, Shh expression was expanded and expressed symmetrically in the matrix cells near the dermal papilla boundary. When taken together with the ultrastructural studies, these data indicate that matrix cells were not growth-arrested, but rather disorganized and blocked in their differentiation. For the IRS, the block appeared to be at the level of GATA-3 expression; for the hair shaft, the block appeared to be past the level of Lef1 expression but before the Wnt-mediated activation of hair-shaft keratin genes.

Bmpr1a null mice were typically frail, and only the mosaic animals survived beyond several days after birth. Consequently, to assess the long-term consequences of ablating BMPR1A expression in skin epithelium, full thickness neonatal skin from Bmpr1a null and wild-type littermate mice were grafted onto the backs of recipient nu/nu mice. Mice with the nu/nu mutation are immunocompromised as they lack T cells and cannot reject the graft. They also lack nearly all external hair shaft formation (Nehls et al. (1994) Nature 372:103-107; Segre et al. (1995) Genomics 28:549-559).

To confirm the uniformity of the conditional targeting of the skin selected for the graft, knockout skin exhibiting spots of organized melanin, but no signs of body hairs, was used. At 24 days after engraftment, hairs were evident only on wild-type grafts. Histologically, these follicles appeared normal. In contrast, the follicles forming in knockout grafts were highly aberrant. In some cases, odd follicle-like structures were found, which had grown downward but displayed only wisps of IRS cells. These follicles lacked a hair shaft and IRS, but possessed a well-developed sebaceous gland. In most cases, cyst-like structures developed. At the light microscopy level, the cells within the cysts appeared morphologically similar to those of embryonic follicles. Ultrastructural analyses revealed a peculiar organization of cells within the cysts. The cysts typically contained invaginations of mesenchymal cells, which were separated from the epithelial cells by a basement membrane. Each cyst was also surrounded by an easily visualized lamina densa, characteristic of an intact basement membrane. On the other side of the basement membrane was a dermal sheath of mesenchymal cells. At the central core of the cysts were often melanin granules. Their presence was of interest as melanin granules are typically taken up by matrix cells as they begin to differentiate to produce the hair shaft. With the exception of a few early stage differentiating cells, the cells within the cysts appeared to be a mixture of matrix, ORS and companion cells, and they displayed numerous mitoses, reflective of a proliferative state.

In cases where follicle from knockout grafted skin had grown downward, the sebaceous glands and ORS were positive for K5, as was the cyst-like structure at the base of the follicle. However, the centers of these follicles were hollow, with no signs of AE15 or AE13 staining. Most cysts expressed K5 toward their periphery and K6 at their centers. Many of the more peripheral cells were also strongly positive for Ki67, confirming the proliferative state of the cysts. Antibodies against GATA-3 weakly labeled a small subset of cells within most cysts. In contrast, many of the cells within the cysts labeled strongly with antibodies against Lef1.

In situ hybridization revealed Shh expression, but in a strikingly unusual pattern, paralleling the number of invaginations of mesenchymal cells seen within each cyst. Closer inspection revealed that Shh appeared to be restricted to a subset of cells maintaining close contact with invaginating dermal papilla-like, mesenchymal cells. Even at this stage, the follicle-like structures from grafts continued to express Wnts, as demonstrated by the marked expression of mRNAs for Wnt10b, a major Wnt of the hair follicle (Reddy et al. (2001) supra). Wnt3, another Wnt prominent in the hair follicle, has been shown to be expressed in the developing limb epithelium of Bmpr1a null animals (Soshnikova et al. (2003) Genes Dev. 17:1963-1968). Thus, expression of both Shh and Wnt ligands persisted in the aberrant knockout follicles.

The corresponding epithelial invaginations surrounding the mesenchymal inlets of cysts were distinguished by anti-β4 staining. In addition, a ring of anti-β4 staining marked the epithelial boundaries of the cysts, which were encased by this membrane of extracellular matrix. On the opposite side were several layers of mesenchymal cells. They differed from a normal dermal sheath in that they were positive for Runx3 and alkaline phosphatase activity. Both Runx3 and alkaline phosphatase are two markers found in dermal condensates including dermal papilla cells (Handjiski et al. (1994) Br. J. Dermatol. 131:303-310; Yamashiro et al. (2002) Gene Expr. Patterns 2:109-112).

Thus, in the absence of BMPR1A, follicles appeared to develop, but the ORS could not sustain its normal architecture in the absence of an IRS and hair shaft. The bulbar structures continued to grow, but reorganized into cyst-like structures. Changes in the biochemistry of the knockout cyst cells appeared to impact indirectly on the organization/specification of both epithelium and mesenchyme.

It was noted that GATA-3 expressed in suprabasal epidermal cells (Kaufman et al. (2003) supra) was downregulated in knockout epidermis as it was in IRS, and Lef1, normally expressed only in embryonic epidermis, was induced in knockout epidermis, albeit at reduced levels relative to knockout follicles. Some epidermal regions in grafted skin were also thicker than their wild-type counterparts, exhibited K6 expression, and increased Ki67 labeling, reflective of hyperproliferative epidermis.

To further explore the molecular defects underlying BMPR1A-related defects in hair shaft differentiation, the Wnt-responsiveness of matrix cells in the Bmpr1a null skin was examined. For these studies, mice conditionally null for BMPR1A and transgenic for TOPGAL, driving β-galactosidase under the control of the Wnt-responsive TOP promoter were generated. Previously, it was shown that in vivo, TOPGAL is faithfully expressed in skin, wherever Lef1 and nuclear β-catenin are present, e.g., as in precortical and cortical cells (DasGupta and Fuchs (1999) supra). Genotyping was conducted to confirm that the desired genetics were successful.

Frozen sections of neonatal skin and cartilage were isolated and subjected to X-gal assays. Wild-type follicles scored positive for TOPGAL activity in cells known to respond to Wnt signaling. In contrast, follicles from Bmpr1a null, TOPGAL-positive mice did not. That TOPGAL was active in knockout animals was confirmed by its presence in tissues where the K14-Cre recombinase was not, e.g., cartilage. The Wnts thought to be responsible for signaling at this stage (Reddy et al. (2001 supra), were still present in Bmpr1a null skin (Soshnikova et al. (2003) supra). These findings indicate that in the absence of BMPR1A, matrix cells are able to express Lef1, but they do not respond to Wnts and hence fail to activate TOPGAL or endogenous, Wnt-responsive hair-specific keratin genes.

In wild-type skin, TOPGAL activity correlated well with cells that exhibit nuclear Lef1 as well as nuclear β-catenin. In knockout skin, even though Lef1 was expressed, nuclear β-catenin was not detected in the corresponding region of developing follicles. These findings indicated that the defect resided in a failure of BMPR1A-deficient follicles to progress to stabilize β-catenin and activate Lef1.

For further analysis, primary keratinocytes isolated from the skins of wild-type and knockout animals were cultured. Polymerase chain reaction was used to confirm the genotype of the cell populations. Two independently derived cultures were prepared for each genotype, and analogous behavior was observed within a particular genotype.

Since keratinocyte cultures produce and secrete high levels of BMP2 and BMP4, it was verified whether the loss of BMPR1A was sufficient to prevent BMP signaling in these cultures. To do so, an antibody specific for the activated form of Smad1, a downstream transcriptional regulator which is phosphorylated and activated upon ligand engagement of BMPR1A (Massague (1998) Annu. Rev. Biochem. 67:753-791) was employed. Wild-type keratinocytes displayed prominent nuclear anti-P-Smad1 staining. In contrast, knockout keratinocytes exhibited little, if any, staining.

It has been shown that cultured neonatal keratinocytes require conditioned medium containing the BMP inhibitor Noggin in order to induce the expression of Lef1 mRNA and protein. In the absence of added Noggin, wild-type keratinocytes displayed little, if any, staining of anti-Lef1. In contrast, knockout keratinocytes displayed strong nuclear anti-Lef1 staining. Western blot analysis confirmed a marked increase in Lef1 expression in the absence of BMPR1A. Overall, these data were consistent with a block in BMP signaling in response to the loss of BMPR1A and further demonstrated that the action of Noggin-conditioned medium on Lef1 expression observed herein in keratinocytes, is mediated through effects on BMP receptor activation.

The ability of knockout follicles to express Lef1 and Wnts and yet fail to activate TOPGAL was indicative of a block in the ability of these cells to respond to Wnt signaling. To explore this possibility further, in vitro studies were conducted using two well-established Wnt-responsive promoters: TOP, containing multimerized Lef1 DNA binding sites (Korinek et al. (1997) Science 275:1784-1787), and HK1, one of the hair-specific keratin promoters containing functional Lef1 DNA binding sites (Zhou et al. (1995) supra; Merrill et al. (2001) supra). As a control, the FOP promoter, harboring mutations in the Lef1 binding sites of TOP, was employed. All promoters were used to drive expression of luciferase (Flash) in wild-type and Bmpr1a null primary keratinocytes.

Under normal media conditions, both wild-type and knockout keratinocytes exhibited very low activation of TOPFlash and HKF1Flash, relative to FOPFlash. In the presence of an N-terminally truncated, highly stable β-catenin, wild-type keratinocytes still showed minimal activation of either TOPFlash or HK1Flash. In marked contrast, however, Bmpr1a null keratinocytes displayed a >5-fold upregulation of TOPFlash and HK1Flash in response to transfected ΔNβ-catenin. The results obtained with Bmpr1a null keratinocytes were similar to those which had been previously observed with wild-type keratinocytes treated with Noggin. The data demonstrate that Bmpr1a null keratinocytes are deficient in Smad activation; these knockout keratinocytes produce Lef1, while wild-type keratinocytes require Noggin treatment for activation; and Wnt-responsive promoter activity in Bmpr1a null keratinocytes can be rescued by transfection with a constitutively stabilized β-catenin.

Not wishing to be bound to a particular mechanism, it appears that BMP signaling is not required for hair progenitor formation as hair progenitor cells form in the absence of BMPR1A. BMPR1A is the only BMP receptor known to be expressed in the hair follicle (Botchkarev et al. (1999) supra), and the follicle abnormalities observed herein were consistent with this. It appears that the molecular block is at the transition between hair shaft and IRS progenitor cells and their differentiation. The BMPR1A pathway has been well-studied biochemically (Massague (1998) supra) and activation results in phosphorylation of Smads (1, 5, and 8), a process which was found defective in the Bmpr1a null keratinocytes provided herein. There are likely a number of precortical genes that are regulated directly by transcriptionally active Smad complexes. The hair keratin genes do not appear to be direct Smad targets, but their expression is completely abrogated in the absence of BMPR1A activation.

Several DNA binding proteins have been implicated in the process, including Lef1, which functions as a positive regulator upon Wnt signaling of the precortical cells (Zhou et al. (1995) supra; DasGupta and Fuchs (1999) supra; Merrill et al. (2001) supra), Hoxcl3, a homeobox gene expressed in matrix, precortical, cortex and cuticle (Jave-Suarex et al. (2001) J. Biol. Chem. 277:3718-3726), and Foxnl, a forkhead/winged-helix transcription factor (Schlake et al. (2000) Gene 256:29-34). When defective, all of these genes yield marked hair follicle phenotypes (van Genderen et al. (1994) Genes Dev. 8:2691-2703; Nehls et al. (1994) supra; Segre et al. (1995) supra; Godwin and Capecchi (1998) Br. J. Dermatol. 131:303-310).

Foxn1 and Hoxc13 are downregulated in Msx2-noggin transgenic follicles (Kulessa et al. (2000) supra). A priori, since Lef1 is upregulated, both in the presence of Noggin (Kulessa et al. (2000) supra) and in the absence of BMPR1A as demonstrated herein, Foxn1 and Hoxc13 may be more likely candidates for BMP control. Hoxc13 and Foxn1 null mutations permit both development of cortex and medulla, whereas Lef1 null mutations act considerably earlier in hair follicle morphogenesis (van Genderen et al. (1994) supra).

The unexpected finding that TOPGAL was not activated in the Bmpr1a null, Lef1-expressing follicle progenitor cells indicated that Lef1-mediated transcriptional activity is compromised in the absence of BMP signaling. Lef1 was clearly functional, as evidenced by the ability of a constitutively stabilized ΔNβ-catenin to rescue TOP as well as hair keratin promoter activity in Bmpr1a-null keratinocytes. In addition, Wnts were still expressed by Bmpr1a-null follicles. Thus, although other factors may be involved, a defect in Wnt responsiveness is sufficient to explain the failure of hair keratin genes to be activated in Bmpr1a-null skin. Foxn1 expression is dependent upon Wnt signaling (Balciunaite et al. (2002) Nat. Immunol. 3:1102-1108) and may explain why Foxnl expression is also downregulated when BMP signaling is blocked.

The results provided herein are of interest in light of studies showing targeted conditional ablation of BMPR1A receptor in the apical ectodermal ridge (Soshnikova et al. (2003) supra). Not only was limb development severely impaired in these animals, Wnt responsiveness was markedly impaired as well. Rescue experiments using gain or loss of function mutations in β-catenin revealed that β-catenin stabilization acts downstream of BMPR1A receptor activation in the apical ectodermal ridge (Soshnikova et al. (2003) supra). Although BMP signaling need not always occur upstream from Wnt signaling (Barrow et al. (2003) Genes Dev. 17:394-409), the results of the present studies indicate that BMP signaling acts upstream from Wnts in hair shaft differentiation, as it does in the apical ectodermal ridge.

1. A method of modulating epithelial stem cell lineage comprising:
a) regulating the expression of Lef1 or a BMP inhibitor and the stability of β-catenin or the expression of a Wnt;
b) regulating the expression or activity of GATA-3; or
c) regulating the expression or activity of bone morphogenetic protein receptor IA
so that epithelial stem cell lineage is inhibited or stimulated.

2. The method of claim 1, wherein the Wnt comprises Wnt1, Wnt2, Wnt 3a, Wnt8a, Wnt 8b, or Wnt10 and the BMP inhibitor comprises noggin, gremlin or chordin.

3. The method of claim 1, wherein bone morphogenetic protein receptor IA stimulates or inhibits inner root sheath or hair shaft formation.

4-7. (canceled)

8. A method of identifying an agent which modulates epithelial stem cell lineage comprising obtaining a test cell which contains:
a) nucleic acid sequences encoding a Wnt, a BMP inhibitor, Lef1, β-catenin and a reporter operably linked to an E-cadherin promoter sequence;
b) a nucleic acid sequence encoding a reporter operably linked to a GATA-3 promoter or a promoter containing a GATA-3 binding site; or
c) a nucleic acid sequence encoding a reporter operably linked to a bone morphogenetic protein receptor IA promoter;
contacting said test cell with at least one test agent and detecting expression of a product of the nucleic acid sequence encoding the reporter in the test cell.

9. The method of claim 8 wherein a decrease in the expression of a product of the nucleic acid sequence encoding the reporter in the test cell contacted with the agent relative to the expression of the product of the nucleic acid sequence encoding the reporter in a test cell not contacted with the agent, indicates that the agent causes a decrease in expression of a product of the nucleic acid sequence encoding E-cadherin, GATA-3, or bone morphogenetic protein receptor IA in the test cell.

10. The method of claim 8 wherein an increase in the expression of a product of the nucleic acid sequence encoding the reporter in the test cell contacted with the agent relative to the expression of the product of the nucleic acid sequence encoding the reporter in a test cell not contacted with the agent, indicates that the agent causes an increase in expression of a product of the nucleic acid sequence encoding E-cadherin, GATA-3, or bone morphogenetic protein receptor IA in the test cell.

11. A method for identifying an agent which modulates inner root sheath or hair shaft formation comprising contacting a test cell, which contains or lacks a functional bone morphogenetic protein receptor IA, with an agent and detecting the phosphorylation state of Smad-1 in the test cell.

12. A method for identifying an agent which stimulates hair shaft formation comprising:
contacting a test cell lacking functional bone morphogenetic protein receptor IA and containing a nucleic acid sequence encoding a reporter operably linked to a Wnt-responsive promoter with an agent;
and detecting expression of a product of the nucleic acid sequence encoding the reporter in the test cell,
wherein an increase in the expression of a product of the nucleic acid sequence encoding the reporter operably linked to a Wnt-responsive promoter in the test cell contacted with the agent relative to the expression of the product of the nucleic acid sequence encoding the reporter in a test cell not contacted with the agent indicates that the agent stimulates hair shaft formation.


SEQUENCE LISTING (12 sequences)

SEQ ID NO: 1 - DNA, 1475 bp, Homo sapiens
aaagcaaatcattcaacgacccccgaccctccgacggcaggagccccccgacctcccaggcggacccgctccctccccgcgcggcgttccgggcccggcgagaggcgcgagcacagccgaggccatggaggtgacggcggaccagccgcgctgggtgagccaccaccaccccgccgtgctcaacgggcagcacccggacacgcaccacccgggcctcagccactcctacatggacgcggcgcagtacccgctgccggaggaggtggatgtgctttttaacatcgacggtcaaggcaaccacgtcccgccctactacggaaactcggtcagggccacggtgcagaggtaccctccgacccaccacgggagccaggtgtgccgcccgcctctgcttcatggatccctaccctggctggacggcggcaaagtcctgggcagccaccacaccgcctccccctggaatctcagccccttctccaagacgtccatccaccacggctccccggggcccctctccgtctaccccccggcctcgtcctcctccttgtcggggggccacgccagcccgcacctcttcaccttcccgcccaccccgccgaaggacgtctccccggacccatcgctgtccaccccaggctccggcggctcggcccggcaggacgagaaagagtgcctcaagtaccaggtgcccctgcccgacagcatgaagctggagtcgtcccactcccgtggcagcatgaccgccctgggtggagcctcctcgtcgacccaccaccccatcaccacctacccgccctacgtgcccgagtacagctccggactcttcccccccagcagcctgctgggcggctcccccaccggcttcggatgcaagtccaggcccaaggcccggtccagcacaggcagggagtgtgtgaactgtggggcaacctcgaccccactgtggcggcgagatggcacgggacactacctgtgcaacgcctgcgggctctatcacaaaatgaacggacagaaccggcccctcattaagcccaagcgaaggctgtctgcagccaggagagcagggacgtcctgtgcgaactgtcagaccaccacaaccacactctggaggaggaatgccaatggggaccctgtctgcaatgcctgtgggctctactacaagcttcacaatattaacagacccctgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagcctgtcctttggaccacaccacccctccagcatggtcaccgccatgggttagagccctgctcgatgctcac

SEQ ID NO: 2 - DNA, 1599 bp, Homo sapiens
atgactcagctatacatttacatcagattattgggagcctatttgttcatcatttctcgtgttcaaggacagaatctggatagtatgcttcatggcactgggatgaaatcagactccgaccagaaaaagtcagaaaatggagtaaccttagcaccagaggataccttgccttttttaaagtgctattgctcagggcactgtccagatgatgctattaataacacatgcataactaatggacattgctttgccatcatagaagaagatgaccagggagaaaccacattagcttcagggtgtatgaaatatgaaggatctgattttcagtgcaaagattctccaaaagcccagctacgccggacaatagaatgttgtcggaccaatttatgtaaccagtatttgcaacccacactgccccctgttgtcataggtccgttttttgatggcagcattcgatggctggttttgctcatttctatggctgtctgcataattgctatgatcatcttctccagctgcttttgttacaaacattattgcaagagcatctcaagcagacgtcgttacaatcgtgatttggaacaggatgaagcatttattccagttggagaatcactaaaagaccttattgaccagtcacaaagttctggtagtgggtctggactacctttattggttcagcgaactattgccaaacagattcagatggtccggcaagttggtaaaggccgatatggagaagtatggatgggcaaatggcgtggcgaaaaagtggcggtgaaagtattctttaccactgaagaagccagctggtttcgagaaacagaaatctaccaaactgtgctaatgcgccatgaaaacatacttggtttcatagcggcagacattaaaggtacaggttcctggactcagctctatttgattactgattaccatgaaaatggatctctctatgacttcctgaaatgtgctacactggacaccagagccctgcttaaattggcttattcagctgcctgtggtctgtgccacctgcacacagaaatttatggcacccaaggaaagcccgcaattgctcatcgagacctaaagagcaaaaacatcctcatcaagaaaaatgggagttgctgcattgctgacctgggccttgctgttaaattcaacagtgacacaaatgaagttgatgtgcccttgaataccagggtgggcaccaaacgctacatggctcccgaagtgctggacgaaagcctgaacaaaaaccacttccagccctacatcatggctgacatctacagcttcggcctaatcatttgggagatggctcgtcgttgtatcacaggagggatcgtggaagaataccaattgccatattacaacatggtaccgagtgatccgtcatacgaagatatgcgtgaggttgtgtgtgtcaaacgtttgcggccaattgtgtctaatcggtggaacagtgatgaatgtctacgagcagttttgaagctaatgtcagaatgctgggcccacaatccagcctccagactcacagcattgagaattaagaagacgcttgccaagatggttgaatcccaagatgtaaaaatctga

SEQ ID NO: 3 - DNA, 459 bp, Homo sapiens
ccggtcagtggaggggcgggaggaggggcgggggtgcgcggggcggggggagaagtcctggagcgggtttgggttgcagtttccttgtgccggggatcctgtcccctactcgccagcgccaggctcctcccccccggcgcggatgacactagaacctccttaagttgcgtcgcgccacagctgtctgcgaacactgagctgcctggcgccgtcttgatactttcagaaagaatgcattccctgtaaaaaaaaaaaaaaaaaatactgagagagggagagagagagagaagaagagagagagacggagggagagcgagacagagcgagcaacgcaaatctgaccgagcaggtcgtacgccgccgcctcctcctcctctctcctcttcgctacccaggttggtactggtgactttttttttttttaagtttgattttttgcccccaaccacttgggaggac

SEQ ID NO: 4 - DNA, 8 bp, Artificial Sequence
  GATA-3 binding site sequence.
agatctta

SEQ ID NO: 5 - DNA, 703 bp, Homo sapiens
agccaggtggctgaaaacccctcttctggaggttaaagcagtttgcgtcctggaaccataggaggtggcagggaattagggatcctgataaggagtgtggggcagatcccaaaaccacctctgtcctctagcattcggttatttttaactatttctaggctgcactgaagtgatgtctcattcttggtgcctaagccattttacacataaatgtgcattcttctcctggtatatctttgtgtgcagaccaaatctttcccatcacctcttttttacttaacggttggaataaccagagagaggctttgtagcctccaagggagcaaactgaggaatcccccttttgctttggctgctgggctgacccgagctggggtgcagaggtgggtgcggcagcgggcaagaagctgctgcccgaggcgtcccggctgaggtccaccgggtatccgtcgctgccggtctgctggcttagtgaagaacaggcacatgcgtctggttgctgttttgttttatataacaaattgctttagggtccgcgaggtggaaggcagcctagagctgagctgcccggagggaggggctgcgggtagggggcagtccgggaccaagggaggccgggagaagggtgcgcatgccaaacctcctcctttgcacctctgagtcgggcacaaacatggatttttttttttaccaggtggtctct

SEQ ID NO: 6 - DNA, 22 bp, Artificial Sequence
  Synthetic oligonucleotide primer.
tcctgcgagcctggctgtcgga

SEQ ID NO: 7 - DNA, 21 bp, Artificial Sequence
  Synthetic oligonucleotide primer.
cctgtagccagctttcataac

SEQ ID NO: 8 - DNA, 21 bp, Artificial Sequence
  Synthetic oligonucleotide primer.
gttgccttgaccatcgatgtt

SEQ ID NO: 9 - DNA, 25 bp, Artificial Sequence
  Synthetic oligonucleotide primer.
caaagaaaataaaaacataagaaac

SEQ ID NO: 10 - DNA, 20 bp, Artificial Sequence
  Synthetic oligonucleotide primer.
tcctattccacggtcgttcg

SEQ ID NO: 11 - DNA, 20 bp, Artificial Sequence
  Synthetic oligonucleotide primer.
agcacctctatagatgaggc

SEQ ID NO: 12 - DNA, 23 bp, Artificial Sequence
  Synthetic oligonucleotide primer.
tactaaggccaaaacaatcactg